(19) United States
(12) Patent Application Publication
(10) Pub. No.: US 2009/0208434 A1
(43) Pub. Date: Aug. 20, 2009

(54) 3-(4-hydroxy-3-methoxyphenyl)-1-(4-hydroxyphenyl)-1-propanone and its use in cosmetic and pharmaceutical preparations

(75) Inventors: Gerhard Schmaus, Hoxter-Bosseborn (DE); Oskar Koch, Gottingen (DE)

Correspondence Address: CONNOLLY BOVE LODGE & HUTZ LLP, 1875 EYE STREET, N.W., SUITE 1100, WASHINGTON, DC, 20006, US

(73) Assignee: SYMRISE GmbH & Co. KG, Holzmiden (DE)

(21) Appl. No.: 12/388,030

(22) Filed: Feb. 18, 2009

(30) Foreign Application Priority Data
Feb. 18, 2008 (DE) 10 2008 009 929.5

(51) Int. Cl. A61K 8/35; A61K 31/12; C07C 49/255

(52) U.S. Cl. 424/59; 514/685; 568/331

(57) ABSTRACT

The present invention concerns applications of the compound of formula 1

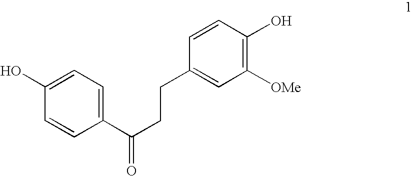

in cosmetic or pharmaceutical preparations.

The present application asserts the benefit of priority to DE 10 2008 009 929.5, filed on Feb. 18, 2008, the disclosure of which is incorporated by reference herein.

The present invention concerns applications of the compound of formula 1

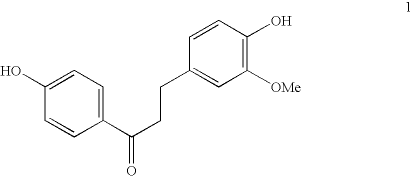

in cosmetic or pharmaceutical preparations.

As a barrier organ in the human organism, the skin, in particular the epidermis, is subject to considerable external effects. Many intrinsic factors (such as genetic predisposition and/or changes associated with the natural ageing process) and extrinsic factors (such as damage to the skin barrier, effect of UV light, irritants or allergens, mechanical effects, e.g. from shaving) can lead to irritation of the skin. Skin irritation, in the context of the present application is understood to be alteration of the skin which in humans or animals triggers sensorial discomfort and/or is characterized by a dry, reddened and/or irritated skin condition. Here the expression sensorial discomfort obviously also covers states of itching or pain. Skin irritation can in particular include phenomenologically different skin conditions: sensitive skin, sore skin, including a sore scalp, vulnerable skin, atopic skin, irritated skin, Rosacea, inflamed skin, which expresses itself according to the degree of seriousness as a reddening of the skin, so-called erythema.

The problem of “sensitive skin” concerns a growing number of adults and children. It is now estimated that up to 50% of the population has a sensitive skin (L. Misery et al., Ann. Dermatol. Venereol. 2005, 132, 425-429). Sensitive skin denotes a skin with a reduced threshold to stimulus from irritants, such as hyperactive and intolerant, but also atopic, skin. In humans with sensitive, sore or vulnerable skin the phenomenon of stinging may be observed. Typical disturbing phenomena associated with the expressions “stinging” or “sensitive skin” are reddening of the skin, tingling, prickling, tightness and burning of the skin and itching. These can be brought about by stimulating ambient conditions such as massage, the effects of surfactants, the effects of other chemical substances such as lactic acid, the effects of the weather such as warmth, cold and dryness, but also damp heat, heat radiation and UV radiation, e.g. the sun, or physiological stress.

A “sore” scalp is also characterized by reddening of the skin, tingling, prickling, burning and itching. Examples of triggers include soap, shampoos or other hair care products, surfactants, hard water with high concentrations of lime and/or mechanical stress. Erythema and hyperseborrhoea (excessive production of sebaceous matter) of the scalp, as well as dandruff are often associated with the phenomena described.

In around 10-20%, and rising, of the population of industrialized nations, atopy is observed. This is a familial oversensitivity of the skin and the mucous membranes to environmental materials, with an increased propensity to develop oversensitivity reactions to substances from the natural world of the immediate type (allergies). Atopy is probably genetically conditioned. Atopy can express itself as atopic dermatitis. Here the skin barrier is damaged, the skin is often irritated and itches.

The erythematous effect of the ultraviolet part of sunlight or artificial radiation is generally known. While radiation with a wavelength of less than 290 nm (the so-called UVC-range), is absorbed by the ozone layer of the earth's atmosphere, radiation in the range between 290 nm and 320 nm, the so-called UVB range, causes an erythema, simple sunburn or even more or less serious burns. Erythematous skin abnormalities also occur as concomitant phenomena in certain skin complaints or irregularities, such as acne, bacterially induced inflammations of the skin, cutaneous reactions, dermographism, general soreness of the skin, creeping eruption, traumatic erysipelas, shingles, frostbite or burns. A particular form of reddening of the skin here is Rosacea. Anti-inflammatory preparations can therefore also be advantageously used to ease reddening of the skin caused by illness and in particular Rosacea, suppress this over a longer period and prevent a reoccurrence.

Skin inflammation, accompanied by redness and itching, can likewise be caused by insect bites. Anti-inflammatories can therefore help to alleviate the effects of an insect bites, such as redness, wealing, itching and painful swelling of the skin, suppress these over a longer period and prevent a reoccurrence.

Intense erythemas also occur in small children in the nappy area, even more so in infants (napkin-area dermatitis). Incontinence, a complaint that affects the elderly in particular, is often associated with erythemas and reddening of the skin as a result of prolonged exposure to moisture and irritants (incontinence dermatitis).

Although in the technical areas mentioned a number of substances which reduce skin irritation are already used, further alternatives are still to be sought.

In the cosmetic and pharmaceutical industry, therefore, there is a constant need for agents that have the effect of reducing skin irritation. In searching for such anti-inflammatories it must be borne in mind that substances used in cosmetic and/or pharmaceutical products must also be
toxicologically safe,
well tolerated by the skin,
stabile (in particular in normal cosmetic and/or pharmaceutical formulations),
preferably low odor or (as far as possible) odorless,
preferably colorless and not discoloring, and
economical to produce (i.e. by using standard methods and/or on the basis of standard precursors).

The search for suitable (active) substances, having one or more of the said characteristics to a sufficient degree, is made harder for a person skilled in the art by the fact that there is not clear dependency between the chemical structure of a substance on the one hand and its anti-inflammatory activity and its stability on the other. Furthermore, there is no predictable correlation between the anti-inflammatory effect, the toxicological safety, the tolerance by the skin and/or the stability.

The problem for the present invention was therefore to provide an anti-inflammatory active substance, which preferably meets one or more, and most preferably all, of the abovementioned auxiliary conditions.

The task of the present invention is solved by the objects of the independent claims. The preferred embodiments are described in the dependent claims and in the description that follows. Here the preferred embodiments according to the invention can also be combined with one another.

For use in accordance with the invention is the compound of formula 1:

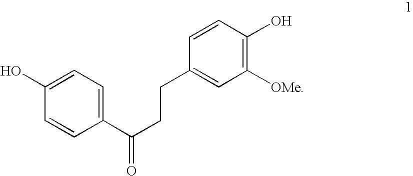

In the context of the present invention and its preferred embodiments, the terms “compound of formula 1” and “3-(4-hydroxy-3-methoxyphenyl)-1-(4-hydroxyphenyl)-1-propanone” also refer to their solvates, preferably hydrates, and salts, preferably phenolate salts.

An object of the present invention thus concerns a cosmetic or pharmaceutical preparation including or comprising the following components:
a) a quantity with an anti-inflammatory effect of a compound of formula 1

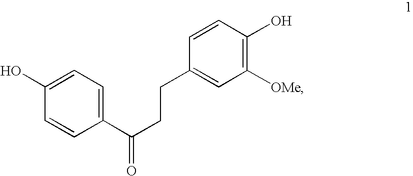

b) if necessary one, two, three, four or more additional compounds with an anti-inflammatory effect,
and
c) one or more cosmetic and/or pharmaceutical base or inactive ingredients or additives that are compatible with components (a) and (b).

A further object of the present invention concerns a compound of formula 1

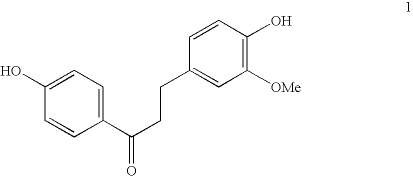

for use as a medicinal product.

A further object of the present invention concerns the use of a compound of formula 1

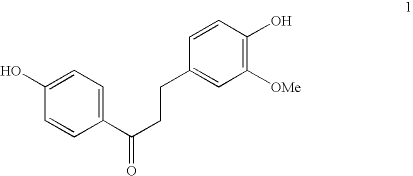

in a quantity with an anti-inflammatory effect in cosmetic preparations.

A further object of the present invention concerns the use of a compound of formula 1

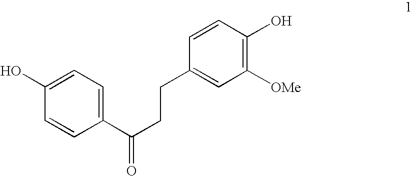

in a quantity with an anti-inflammatory effect for the production of a medicinal product to treat or prevent inflammation.

A further object of the present invention concerns the use of a compound of formula 1

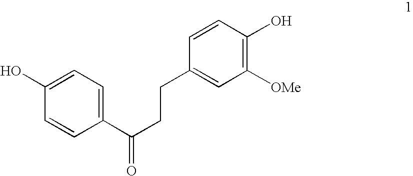

in a quantity with an anti-inflammatory effect for the treatment or prevention of inflammation.

A further object of the present invention concerns a use of a quantity with an anti-inflammatory effect of a compound of formula 1

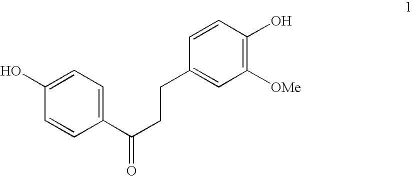

or an inventive cosmetic or pharmaceutical preparation for reducing, eliminating or suppressing an inflammatory effect of one, two, three, four or more compounds on the human or animal skin.

A further object of the present invention concerns a method for cosmetic treatment or prevention of inflammatory processes of the skin including or comprising the topical application of a quantity with an anti-inflammatory effect of the inventive cosmetic or pharmaceutical preparation to the skin of a human or an animal's body.

A further object of the present invention concerns a method for the treatment or prevention of an inflammatory effect of one, two, three, four or more compounds on the skin of a human or an animal's body, including or comprising the following steps:
a) provision of one, two, three, four or more compounds that have an inflammatory effect on the skin,
b) provision of a compound of formula 1

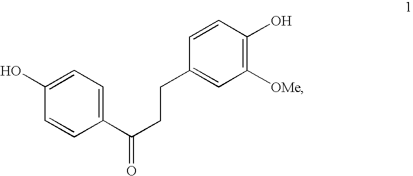

c) if necessary provision of one, two, three, four or more base or inactive ingredients and/or additives for cosmetic or pharmaceutical preparations and
d) mixing of the compounds from step a) with the compound of formula 1 from step b) and if necessary the base or inactive ingredients and/or additives from step c) to form a preparation,
   in which one or more compound(s) from step a) is/are included in a quantity in relation to the finished preparation, which in a preparation without the compound of formula 1 have an inflammatory effect on human or animal skin and
   in which the compound of formula 1 is included in a quantity that is sufficient to reduce, eliminate or suppress the inflammatory effect of the compounds from step a) on the skin.

A further object of the present invention concerns an aromatic substance composition including or comprising
a) a quantity of an aromatic substance or mixture of aromatic substances with a sensorial effect,
b) a quantity with an anti-inflammatory effect of a compound of formula 1

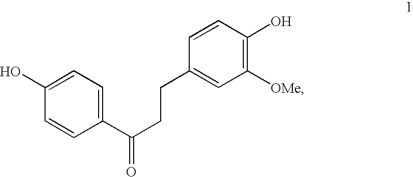

c) if necessary one, two, three, four or more additional compounds with an anti-inflammatory effect and
d) if necessary one or more base or inactive ingredients and/or additives.

The present invention is based on the surprising realization that the compound of formula 1 has outstanding anti-inflammatory characteristics and can therefore be used in an outstanding manner, in particular in a quantity with an anti-inflammatory effect, in cosmetic or pharmaceutical preparations. Thus the compound of formula 1 can be used in order to treat inflammations or to prevent these. Such inflammations are preferably triggered by extrinsic factors, preferably by mechanical irritation, chemical irritation and irritation from sunlight; and/or intrinsic factors, preferably genetic factors and natural ageing processes.

The present invention is also based on the surprising realization that by using the compound of formula 1 in combination with one, two, three, four or more compounds, which have an inflammatory effect, preferably irritation and/or reddening of human or animal skin, the inflammatory effect of these compounds can at least in part be reduced, eliminated or suppressed. Consequently, in accordance with the invention compounds with an inflammatory effect on human or animal skin can also be used in cosmetic or pharmaceutical preparations.

Investigations of the chemical characteristics have shown that the compound of formula 1 has the distinction of having high stability, in particular high temperature stability, high stability in a broad pH range and high photostability, in which it can be used in an outstanding manner in the most varied of cosmetic preparations and pharmaceutical products (medicinal products, medical devices). The inventive compound of formula 1 in its pure form is constituted by a white solid, which dissolves in a colorless manner in cosmetic and/or pharmaceutical base or inactive ingredients or additives, preferably excipients. Furthermore, once the inventive compound of formula 1 had been incorporated into inventive cosmetic and pharmaceutical preparations no discoloration of this preparation was detected.

In DE 10 2007 035 139.0 (Symrise GmbH & Co. KG) 3-(4-hydroxy-3-methoxyphenyl)-1-(4-hydroxyphenyl)-1-propanone, corresponding to the compound of formula 1 of the present invention, a method for the manufacture thereof and the use of 3-(4-hydroxy-3-methoxyphenyl)-1-(4-hydroxyphenyl)-1-propanone as an anti-microbial active substance are described. DE 10 2007 035 139.0 does not disclose that 3-(4-hydroxy-3-methoxyphenyl)-1-(4-hydroxyphenyl)-1-propanone, corresponding to the compound of formula 1 of the present invention, is effective as an anti-inflammatory.

In U.S. Provisional Application 60/951,741 (Symrise GmbH & Co. KG) 3-(4-hydroxy-3-methoxyphenyl)-1-(4-hydroxyphenyl)-1-propanone, corresponding to the compound of formula 1 of the present invention, a method for the manufacture thereof and the use of 3-(4-hydroxy-3-methoxyphenyl)-1-(4-hydroxyphenyl)-1-propanone as an anti-oxidant are described. U.S. 60/951,741 does not disclose that 3-(4-hydroxy-3-methoxyphenyl)-1-(4-hydroxyphenyl)-1-propanone, corresponding to the compound of formula 1 of the present invention, is effective as an anti-inflammatory.

The strong anti-inflammatory effect of the inventive compound of formula 1 is based inter alia on its characteristic of inhibiting the upregulation of inflammatory mediators, including interleukins (in particular IL-1 and IL-6), PGE-2 (prostaglandin E2) and in particular TNF-alpha (tumor necrosis factor alpha) caused by extrinsic and intrinsic factors, according to the dose and even at low concentrations. It can therefore be used as an outstanding alternative or as an addition to other already well-known anti-inflammatory compounds (active substances) in cosmetic and pharmaceutical preparations or similar as an anti-inflammatory active substance.

Until now nothing was known of the anti-inflammatory characteristics, which are described here for the first time and are thus inventive, of 3-(4-hydroxy-3-methoxyphenyl)-1-(4-hydroxyphenyl)-1-propanone and its use in inventive cosmetic and pharmaceutical, preferably dermatological preparations for the prevention of skin irritation and/or the treatment of inflammations, preferably skin irritations and reddening of the skin (erythemas) as well as other undesirable alterations associated with inflammatory processes.

The term “inflammations” (irritations) in the context of the present invention means irritation and/or reddening of the skin (erythemas) as well as other (secondary) undesirable alterations associated with inflammatory processes.

The term “skin” according to the present invention also includes the mucous membrane.

In the context of this invention “skin irritation” is understood to be alteration of the skin which in humans or animals triggers sensorial discomfort and/or is characterized by a dry, reddened and/or irritated skin condition. Here the expression “sensorial discomfort” obviously also covers states of itching or pain. Skin irritation according to the invention can in particular include phenomenologically different skin conditions: sensitive skin, sore skin, including a sore scalp, vulnerable skin, atopic skin, irritated skin, Rosacea, inflamed skin, which expresses itself according to the degree of seriousness as a reddening of the skin, so-called erythema.

The term “reddening of the skin” or “erythema” in connection with the present invention normally means a reddening of the skin caused by ultraviolet radiation (from sunlight of course but also from artificial radiation) on the skin, but also the concomitant phenomena that occur in certain skin complaints or irregularities.

For example, the “reddening of the skin” in accordance with the invention is also constituted by the typical cutaneous eruption that is a feature of acne, which results from the associated inflammatory processes and affects the wellbeing of the person concerned even in minor cases. Other skin abnormalities, which are accompanied by temporary or lasting reddening of the skin in accordance with the invention include, for example, preferably bacterially induced inflammations of the skin, cutaneous reactions, dermographism, general soreness of the skin, creeping eruption, traumatic erysipelas, shingles, frostbite or burns. A particular form of reddening of the skin here is Rosacea. Rosacea [from the Latin rosaceus >rose-colored<], also known as couperose, is a chronic complaint that usually affects the facial skin of middle- and old-aged people, particularly around the nose, the forehead and the cheeks (butterfly patches). Through the dilatation of the superficial cutaneous vessels sometimes dark red vascular congestion, dilatation of the vessels, depending on the form also cutaneous scaling, episodic formation of papules or pustules and (usually only in men) subsequently the formation of a rhinophyma can occur. Reddening of the skin in general and Rosacea in particular constitute a high physical and psychological burden for the person affected. For the observer also they are usually perceived as unattractive, pathological and un-aesthetic.

Inflammations of the skin (skin irritations) in accordance with the invention, accompanied by reddening and itching, can also be caused by insect bites. The reaction to an insect bite is usually a harmless localized alteration of the skin as a result of a bite, in particular from gnats, houseflies, lice, fleas and bugs. The skin alteration itself usually comprises itching, and also painful swellings of the skin. Bites from bees, wasps, hornets and bumble-bees, in which the effect comes from toxins and allergens, depending on the sensitivity of the organism and the site of the bite can lead to reddening, the formation of weals and painful swelling at the site of the bite.

Intense reddening of the skin (erythemas) in accordance with the invention also occurs in small children in the nappy area, even more so in infants (napkin-area dermatitis). Incontinence, a complaint that affects the elderly in particular, is often associated with erythemas and reddening of the skin as a result of prolonged exposure to moisture and irritants (incontinence dermatitis).

The term “skin irritation reducing effect” in the context of this invention means the lessening, reduction, ceasing or prevention of skin irritations and/or reddening, in particular as described above. Here the skin irritation and/or skin reddening reducing effect are in especially based on calming the skin, inhibiting irritation and/or alleviating redness.

Inventive cosmetic or pharmaceutical preparations are preferred, wherein the preparation also
d) includes an effective quantity of one, two, three, four or more UV filters, so that the sun protection factor of the cosmetic or pharmaceutical preparation is >2.

A further preferred embodiment of an inventive cosmetic or pharmaceutical preparation is characterized in that the preparation also includes
e) one, two, three, four or more compounds in a quantity in relation to the finished preparation, which in a preparation without the compound of formula 1 has an inflammatory effect on human or animal skin,
   in which the quantity of the compound of formula 1 with reference to the finished preparation, is sufficient to reduce, eliminate or suppress the inflammatory effect of the compounds from step e) on the skin.

In the context of the present invention the one, two, three, four or more compounds from feature e), which have an inflammatory effect on human or animal skin (potentially irritating compounds), are normally inorganic or organic compounds of synthetic or natural origin, which can be used in cosmetic or pharmaceutical preparations and which, depending on the concentration in which they are used and the skin condition, have a potential for irritation. Such skin irritating compounds preferably have the R-phrases R36, R37 and/or R38 in accordance with the German Chemical Act.

Compounds which in accordance with the invention can cause an inflammation of the skin can normally be found in the most varied of compound groups, such as detergents, agents to improve the suppleness of the skin, agents for treating acne, skin lighteners, preservatives, excipients and aromatic substances.

Compounds which, in accordance with the invention, can cause an inflammation of the skin are preferably selected from the groups consisting of
detergents, preferably sodium lauryl sulfate or soaps;
agents to improve the suppleness of the skin, preferably aliphatic and aromatic alpha-hydroxy acids, beta-hydroxy acids or keto acids, preferably glycolic acid, lactic acid and salicylic acid, and derivatives thereof, which are preferably selected from the group comprising amides, lactones, anhydrides and esters, and likewise their organic and inorganic salts;
agents for treating acne, preferably peroxide, more preferably benzoyl peroxide; salicylic acid; resorcinol; and retinol derivates, preferably retinoic acid, retinaldehyde, retinol, retinyl acetate and retinyl palmitate;
skin lighteners, preferably hydroquinone; kojic acid; resorcinol and derivatives of this;
preservatives, preferably parabens, preferably isobutylparaben and propylparaben; phenoxyethanol; and
urea.

The compounds mentioned above, which in accordance with the invention cause an inflammation of the skin, are used in inventive cosmetic or pharmaceutical preparations preferably in a concentration in which, without the use of the compound of formula 1 they cause an inflammation of human or animal skin.

Table 1 below shows the normal dosage information for selected potentially irritating compounds in cosmetic formulations.

| | Dosage details in |
|---|---|
| | relation to the finished |
| | cosmetic compounds [% |
| Potentially irritating compound | by weight] |
| Glycolic acid | 0.5-5.0 |
| Lactic acid | 0.5-5.0 |
| Salicylic acid | 0.5-5.0 |
| Retinoic acid | 0.1-1 |
| Retinol | 0.1-1 |
| Retinyl palmitate | 0.1-1 |
| Hydroquinone | 0.1-1 |
| Kojic acid | 0.1-1 |
| Resorcinol | 0.1-1 |
| Benzoyl peroxide | 0.1-5 |
| Phenoxyethanol | 0.1-5 |
| Propylparaben | 0.1-0.5 |
| Isobutylparaben | 0.1-0.5 |

A further preferred embodiment of the inventive cosmetic or pharmaceutical preparation is characterized in that the preparation is selected from the group comprising liquid formulations, preferably cleaning solutions or impregnating liquids; semi-solid formulations, preferably ointments and crèmes.

Since in the context of the present invention it is a case of the application of the compound of formula 1 or cosmetic preparations or pharmaceutical, preferably dermatological, preparations including the compound of formula 1 for the treatment or prevention of inflammation of the skin, respective application is in each case preferably topical on the skin, in particular the mucous membrane of the human or animal body. In accordance with the invention in this way the inflammations of the skin, preferably skin irritations and reddening of the skin (erythemas), which may inter alia be accompanied by itching and the formation of weals, caused by extrinsic or intrinsic factors, are either inhibited and/or completely prevented.

The inventive pharmaceutical preparation (medicinal product or medical device) can be used in the field of human or veterinary medicine against a number of complaints, such as urticaria, contact dermatitis, Rosacea, atopy and generally all inflammatory processes, including gingival irritations such as peridontal diseases.

The concentration of the inventive compound of formula 1 used in an inventive cosmetic and pharmaceutical preparation or in an inventive pharmaceutical composition is normally here in the range from 0.001 to 20% by weight, preferably in the range from 0.001 to 10% by weight, preferentially in the range from 0.005 to 5% by weight and with particular preference in the range from 0.01 to 1.0% by weight, in each case in relation to the total weight of the cosmetic or pharmaceutical preparation.

The compound of formula 1 itself was not sensitizing and did not cause irritation at a test concentration of 50% by weight. The inventive compound of formula 1 with an anti-inflammatory effect can also be used as a component of inventive scent compositions (odorous substance compositions) and for example to give a perfumed finished product an anti-inflammatory effect. Since the proportion of perfume in a cosmetic finished product is often around 1% by weight, a scent containing the inventive compound of formula 1, will preferably include or be comprised approximately 1-50% by weight of the compound of formula 1.

It has proven particularly advantageous for the compound of formula 1 to have a mild, pleasant, characteristic odor of vanilla pods; for this characteristic predestines it in particular for use as an anti-inflammatory active substance in a scent composition. Furthermore, because of its structure, the compound has a stronger adherence to the skin and hair in which in particular also for applications in rinse off products for the care and treatment of the scalp and the hair a particularly sustainable and long-lasting anti-inflammatory effect can be achieved.

The inventive 3-(4-hydroxy-3-methoxyphenyl)-1-(4-hydroxyphenyl)-1-propanone with an anti-inflammatory effect can be further processed with one or more pharmaceutically and/or cosmetically acceptable base or inactive ingredients or additives, preferably to form an inventive preparation in solid form, in which the compound of formula 1 preferably has a solid excipient added and is then dried using a suitable process. Pharmaceutically or cosmetically compatible or acceptable base or inactive ingredients and additives, in particular also excipients, are those which for the human or animal organisms on which they are to be used at least have no toxic effects.

The inventive 3-(4-hydroxy-3-methoxyphenyl)-1-(4-hydroxyphenyl)-1-propanone with an anti-inflammatory effect can also be further processed into an inventive diluted preparation in liquid form by optionally adding to the mixture a pharmaceutically and/or cosmetically acceptable solvent such as neutral oil, mineral oil, silicon oil, vegetable oils, fatty alcohols, fatty acid esters, ethanol, 1,2-propylene glycol, 1,3-butylene glycol, 1,2-pentanediol and water as well as mixtures of two or more of the said solvents. Such inventively manufactured preparations can be easily processed further for cosmetic purposes. If necessary these inventive preparations can be produced with the addition of a solubilizer, preservative or antioxidant.

Furthermore, the inventive 3-(4-hydroxy-3-methoxyphenyl)-1-(4-hydroxyphenyl)-1-propanone with an anti-inflammatory effect or the liquid or solid preparation containing this can also be further processed by encapsulation. In accordance with the invention 3-(4-hydroxy-3-methoxyphenyl)-1-(4-hydroxyphenyl)-1-propanone with an anti-inflammatory effect and the liquid or solid preparation containing this is encapsulated with a solid shell material, which is preferably selected from starch, decomposed or chemically or physically modified starch (in particular dextrin and maltodextrin), gelatines, wax materials, liposomes, gum arabic, agar-agar, ghatti gum, gellan gum, modified and unmodified celluloses, pullulan, curdlan, carageenan, alginic acid, alginate, pectin, inulin, xanthan gum and mixtures of two or more of said substances.

Major areas of application for 3-(4-hydroxy-3-methoxyphenyl)-1-(4-hydroxyphenyl)-1-propanone with an anti-inflammatory effect are cosmetic (topical) preparations, which apart from the presence of the compound of formula 1 have a normal composition and serve to provide cosmetic light protection, treatment, care and cleaning of the skin and/or hair or as a make-up product in decorative cosmetics. Accordingly, such preparations can, for example, exist in the form of cleansing agents, such as e.g. soap, synthetic detergent, liquid washing, shower and bath preparation, a skin care product such as e.g. an emulsion (as a solution, dispersion, suspension; crème lotion or milk, depending on the method of manufacture and contents of the W/O, O/W or multiple emulsion, PIT emulsion, emulsion foam, micro- or nano-emulsion, Pickering emulsion type), ointment, paste, gel (including hydro-, hydrodispersion-, oil gel), alcoholic or aqueous/alcoholic solution, oil, toner, balsam, serum, powder, wipe, eau de toilette, eau de Cologne, perfume, wax, including forms of administration such as a pencil, roll-on, (pump-)spray, aerosol (foaming, non-foaming or post-foaming), skin-care product (as described above), a foot care product (including keratolytic, deodorant), an insect repellent, a sunscreen, a self-tanning agent and/or aftersun preparation, a skin care product as a shaving product, or aftershave, a depilatory agent, a hair care product such as e.g. shampoo (including shampoo for normal hair, for dry, stressed (damaged) hair, 2-in-1 shampoo, anti-dandruff shampoo, baby shampoo, shampoo for dry scalps, concentrated shampoo), conditioner, hair tonic, hair water, hair rinse, styling crème, pomade, perm and setting lotion, hair smoothing agent (detangling agent, relaxer), hair spray, styling aids (e.g. gel or wax), a blonding agent, hair dye such as e.g. temporary direct-dyeing hair dye, semi-permanent hair dye, permanent hair dye, skin care products such as e.g. decorative body care products, such as e.g. nail care products (nail varnish and nail varnish remover), decorative cosmetics (such as powders, eye shadow, eyeliner pencils), skin care products such as deodorants and/or antiperspirants, mouthwashes and oral douches.

The (in particular topical) cosmetic or pharmaceutical, preferably dermatological preparations can contain one, two, three, four or more base or inactive ingredients or additives, as normally used in such preparations, for example selected from the group comprising sunscreens, preservatives, bactericides, fungicides, virucides, coolants, insect repellents (e.g. DEET, IR 3225), plant extracts, plant components, other anti-anti-inflammatory agents, substances to accelerate wound healing (e.g. chitin or chitosan and derivatives thereof), film formers (e.g. polyvinyl pyrrolidones or chitosan or derivatives thereof), antioxidants, vitamins, 2-hydroxycarboxylic acids (e.g. citric acid, malic acid, L-, D- or DL-lactic acid), skin coloring agents (e.g. walnut extracts or dihydroxyacetone), hair growth acceleration and inhibiting agents, skin care agents (e.g. cholesterol, ceramides, pseudoceramides), softening, moisturizing and/or moisture retention agents, fats, oils, saturated fatty acids, monounsaturated or polyunsaturated fatty acids, alpha-hydroxy acids, polyhydroxy fatty acids or derivatives thereof, waxes or other conventional constituents of a cosmetic or dermatological formulation such as alcohols, polyols, polymers, foam stabilisers, electrolytes, organic solvents, silicone derivatives or chelating agents (e.g. ethylene diamine tetraacetic acid and derivatives), anti-dandruff agents (e.g. climbazole, ketoconazole, piroctone olamine, zinc pyrithione), hair care products, perfumes, substances to prevent foaming, dyes, pigments having a coloring effect (advantageously silicon dioxide, aluminum silicate, such as e.g. bentonites, polysaccharides or derivatives thereof, e.g. hyaluronic acid, guar gum, xanthan gum, hydroxypropyl methyl cellulose, or allulose derivatives, particularly advantageously polyacrylates, such as carbopols or polyurethane), surface-active agents and emulsifiers.

The quantities to be used in each case of cosmetic or pharmaceutical (possibly dermatological) base or inactive ingredients or additives and scents can, depending on the type of product concerned be easily determined by a person skilled in the art by simple testing.

In accordance with the invention the cosmetic and pharmaceutical preparations and scent compositions include possibly one, two, three, four or more additional compounds with an anti-inflammatory effect. Here all the usual active substances employed for reducing skin irritation and/or reddening of the skin can be used. Particular preference is, however, for combinations with one, two, three, four or more additional compounds with an anti-inflammatory effect selected from the group of the following compounds: steroidal anti-inflammatory substances of the corticosteroidal type, preferably hydrocortisone, hydrocortisone derivates such as hydrocortisone-17-butyrate, dexamethasone, dexamethasone phosphate, methyl prednisolone or cortisone and other steroidal anti-inflammatories; non-steroidal anti-inflammatories, preferably Oxicams such as piroxicam or tenoxicam; salicylates such as aspirin, disalcid, Solprin or Fendosal; acetic acid derivatives such as diclofenac, fenclofenac, indomethacin, sulindac, tolmetin, or clindanac; fenamates such as mefenamic, meclofenamic, flufenamic or niflumic; propionic acid derivatives such as ibuprofen, naproxen, benoxaprofen or pyrazols such as phenylbutazone, oxyphenylbutazone, febrazone or azapropazone; natural anti-inflammatories or reddening and/or itching alleviators, preferably plant extracts, special highly active plant extract fractions and highly pure active substances isolated from plant extracts, particularly preferred are extracts, fractions and active substances from camomile, ginger, aloe vera, commiphora species, licorice (Glycyrrhiza) species such as Glycyrrhiza glabra and Glycyrrhiza inflata, rubia species, willow, willowherb, oats, marigold, arnica, St. John's wort, goat's leaf, rosemary, melissa, maypop, witch hazel, kudzu, dianthus or echinacea as well as pure substances isolated from these, preferably bisabolol, apigenin, apigenin-7-glucoside, rosmarinic acid, boswellia acid, phytosterols, glycyrrhetic acid, glabridin, lichochalcone A and anthranilic acid amides such as in particular avenanthramides or dianthramides; naturally occurring anthranilic acid derivatives, preferably of oats or carnation cloves (i.e. Dianthus species); fully or partially synthetically manufactured anthranilic acid amides, preferably dihydroavenanthramide D (Symrise GmbH/Co KG); naturally occurring components of licorice and/or fully or partially synthetically manufactured compounds, preferably tetrahydro-licochalcone A.

The bisabolol used in connection with the present invention can be of natural or synthetic origin, preferably it is “alpha-bisabolol” that is involved. The term “alpha-bisabolol” here includes in the context of this document (+)-alpha-bisabolol, (−)-alpha-bisabolol, (+)-epi-alpha-bisabolol and (−)-epi-alpha-bisabolol as well as mixtures of two, three or all of the said isomers of alpha-bisabolol. In particular the term “alpha-bisabolol” covers racemic mixtures of (+/−)-alpha-bisabolol and/or (+/−)-epi-alpha-bisabolol. The bisabolol used is preferably a synthetically manufactured or natural (−)-alpha-bisabolol and/or synthetic isomer mixed alpha-bisabolol. Where natural (−)-alpha-bisabolol is used, this can also be used as a component of an essential oil or a plant extract or a fraction thereof, for example as a component of (fraction of) camomile oils or extracts or Vanillosmopsis (in particular Vanillosmopsis erythropappa or Vanillosmopsis arborea). Synthetic alpha-bisabolol is, for example, available under the name “Dragosantol” from Symrise GmbH & Co. KG.

In the case of extract of ginger it is preferably a case of extracts of fresh or dried root ginger prepared by extraction with methanol, ethanol, isopropanol, acetone, ethyl acetate, carbon dioxide (CO₂), hexane, dichloromethane, chloroform or other solvents or mixtures of solvents of similar polarity. The extracts are characterized by the presence of quantities that are effective in reducing skin irritation of, for example, gingerols, shogaols, ginger diols, dehydrogingerdiones and/or paradols.

The quantity of the one, two, three, four or more additional compounds with an anti-inflammatory effect (anti-irritants) in the inventive preparations is preferably a total of 0.0001 to 20% by weight, with particular preference for 0.0001-10% by weight, and most particularly 0.001-5% by weight, with reference to the total weight of the preparation.

Inventive cosmetic and pharmaceutical, preferably dermatological, preparations can furthermore contain one, two, three, four or more antioxidants, in which all the usual antioxidants that are suitable for cosmetic and pharmaceutical, preferably dermatological, applications can be used. Advantageously the one, two, three, four or more antioxidants are selected from the group comprising amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. alpha-carotene, beta-carotene, lycopene) and derivatives thereof, chlorogenic acid and derivatives thereof, lipoic acid and derivatives thereof (e.g. dihydrolipoic acid), aurothioglucose, propyl thiouracil and other thiols (e.g. thioredoxine, glutathione, cysteine, cystine, cystamine and glycosyl, N-acetyl, methyl, ethyl, propyl, amyl, butyl and lauryl, palmitoyl, oleyl, gamma-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), (metal) chelators e.g. alpha-hydroxy fatty acids, palmitic acid, phytic acid, lactoferrin, alpha-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. gamma-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate, ascorbyl glycoside, e.g. 6-O-acyl-2-O-α-D-glucopyranosyl-L-ascorbic acid, 6-O-acyl-2-O-β-D-glucopyranosyl-L-ascorbic acid, 2-O-α-D-glucopyranosyl-L-ascorbic acid or 2-O-β-D-glucopyranosyl-L-ascorbic acid), tocopherols and derivatives (e.g. vitamin E acetate), vitamin A and derivatives (vitamin A palmitate) and coniferyl benzoate of benzoic resin, rutinic acid and derivatives thereof, alpha-glucosyl rutin, quercetin and derivatives thereof, rosmarinic acid, carnosol, carnosolic acid, resveratrol, caffeic acid and derivatives thereof, sinapic acid and derivatives thereof, ferulic acid and derivatives thereof, furfuryliden glucitol, curcuminoids, butyl hydroxytoluene, butyl hydroxyanisole, nordihydroguaiac acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO₄), selenium and derivatives thereof (e.g. selenium methionine), stilbenes and derivatives thereof (e.g. stilbene oxide, trans-stilbene oxide) and the derivatives (salts, esters, ethers, sugars, nucleotides, nucleosides, peptides and lipids) of these cited active ingredients or extracts or fractions of plants having an antioxidant effect, such as e.g. green tea, rooibos, honeybush, grape, rosemary, sage, melissa, thyme, lavender, olive, oats, cocoa, ginkgo, ginseng, licorice, goat leaf, sophora, pueraria, Pinus, Citrus, Phyllanthus emblica or St. John's wort.

The total quantity of antioxidants (one or more compounds) in the inventive preparations is preferably 0.01 to 20% by weight, with particular preference for 0.05-10% by weight, and most particularly 0.2-5% by weight, with reference to the total weight of the preparation.

Advantageously the inventive cosmetic and pharmaceutical, preferably dermatological, preparations also contains one, two, three, four or more UV filters, preferably selected from the group comprising UVA filters, UVB filters and or inorganic pigments. Here the inventive preparations can exist in various formulations, as normally used in sunscreen preparations to protect the skin and hair from ultraviolet radiation. Thus they may preferably form a solution, an emulsion of the water-in-oil type (W/O) or the oil-in-water type (O/W), or a multiple emulsion, for example of the water-in-oil-in-water type (WIOIW), a gel, a hydrodispersion, a solid pencil or also an aerosol. Here the total quantity of UV filter substances is from 0.01% by weight to 40% by weight, preferably 0.1% by weight to 10% by weight, in particular 1.0 to 5.0% by weight, with reference to the total weight of the preparations.

Advantageous UV filters are, for example: p-aminobenzoic acid, p-aminobenzoic acid ethyl ester (25 mol) ethoxylated, p-dimethylaminobenzoic acid-2-ethylhexyl ester; p-aminobenzoic acid ethyl ester (2 mol) N-propoxylated, p-aminobenzoic acid glycerol ester, salicylic acid homomethyl ester (Homosalate) (Neo Heliopan®HMS), salicylic acid-2-ethylhexyl ester (Neo Heliopan®OS), triethanolamine salicylate, 4-isopropyl benzyl salicylate, anthranilic acid menthyl ester (Neo Heliopan®MA), diisopropyl cinnamic acid ethyl ester, p-methoxycinnamic acid-2-ethylhexyl ester (Neo Heliopan®MA), diisopropyl cinnamic acid methyl ester, p-methoxycinnamic acid isoamyl ester (Neo Heliopan®E 1000), p-methoxycinnamic acid diethanolamine salt, p-methoxycinnamic acid isopropyl ester, 2-ethylhexyl-2-cyano-3,3-diphenyl acrylate (Neo Heliopan®303), ethyl-2-cyano-3,3′-diphenyl acrylate, 2-phenylbenzimidazolesulfonic acid and salts (Neo Heliopan®hydro), 3-(4′-trimethylammonium)-benzylidene-bornan-2-one methylsulfate, terephthalylidene-dibornanesulfonic acid and salts (Mexoryl®SX), 4-t-butyl-4′-methoxy-dibenzoylmethane (Avobenzone)/(Neo Heliopan®357), β-imidazole-4(5)-acrylic acid (Urocanic acid), 2-hydroxy-4-methoxybenzophenone (Neo Heliopan®BB), 2-hydroxy-4-methoxybenzophenone-5-sulfonic acid, dihydroxy-4-methoxybenzophenone, 2,4-dihydroxybenzophenone, tetrahydroxybenzophenone, 2,2′-dihydroxy-4,4′-dimethoxybenzophenone, 2-hydroxy-4-n-octoxybenzophenone, 2-hydroxy-4-methoxy-4′-methylbenzophenone, 3-(4′-sulfo)benzylidene-bornan-2-one and salts, 3-(4′-methylbenzylidene)-d,l-camphor (Neo Heliopan®MBC), 3-benzylidene-d,l-camphor, 4-isopropyldibenzoylmethane, 2,4,6-trianilino-(p-carbo-2′-ethylhexyl-1′-ox y)-1,3,5-triazine, phenylene-bis-benzimidazyl-tetrasulfonic acid disodium salt (Neo Heliopan®AP), 2,2′-(1,4-phenylene)-bis-(1H-benzimidazole-4,6-disulfonic acid), monosodium salt, N-[(2 and 4)-[2-(oxoborn-3-ylidene)methyl]benzyl]-acrylamide-polymer, phenol, 2-(2H-benzotriazol-2-yl)-4-methyl-6-(2-methyl-3(1,3,3,3-tetr amethyl-1-(trimethylsilyl)-oxy)-disiloxyanyl)-propyl), (Mexoryl®XL), bis-(2-ethylhexyl) 4,4′-[(6-[4-(1,1-dimethyl)-aminocarbonyl)-phenylamino]-1,3,5-triazine-2,4-diyl)diimino]-bis-(benzoate) (Uvasorb®HEB), 2,2′-methylene-bis-(6-(2H-benztriazol-2-yl)-4-1,1,3,3-tetramethyl butyl)-phenol), (Tinosorb®M), 2,4-bis-[4-(2-ethylhexyloxy)-2-hyd roxyphenyl]-1,3,5-triazine, benzylidene malonate-polysiloxane (Parsol®SLX), glycerylethyl hexanoate-dimethoxycinnamate, disodium 2,2′-dihydroxy-4,4′-dimethoxy-5,5′-disulfo-benzophenone, dipropylene glycol salicylate, sodium hydroxymethoxybenzophenonesulfonate, tris (2-ethylhexyl) 4,4′,4-(1,3,5-triazine-2,4,6-triyltriimino)-tris-benzoate (Uvinul®T150), 2,4-bis-[{(4-(2-ethyl-hexyloxy)-2-hydroxy}-phenyl]-6-(4-methoxyphenyl)-1,3,5-triazine, (Tinosorb®S), 2,4-bis-[{(4-(3-sulfonato)-2-hydroxy-propyloxy)-2-hydroxy}-phenyl]-6-(4-methoxyphenyl)-1,3,5-triazine sodium salt, 2,4-bis-[{(3-(2-propyloxy)-2-hydroxy-propyloxy)-2-hydroxy}-phenyl]-6-(4-methoxy-phenyl)-1,3,5-triazine, 2,4-bis-[{4-(2-ethyl-hexyloxy)-2-hydroxy}-phenyl]-6-[4-(2-methoxyethyl-carbonyl)-phenylamino]-1,3,5-triazine, 2,4-bis-[{4-(3-(2-propyloxy)-2-hydroxy-propyloxy)-2-hydroxy}-phenyl]-6-[4-(2-ethylcarboxyl)-phenylamino]-1,3,5-triazine, 2,4-bis-[{4-(2-ethyl-hexyloxy)-2-hydroxy}-phenyl]-6-(1-methyl-pyrrol-2-yl-)-1,3,5-triazine, 2,4-bis-[{4-tris-(trimethylsiloxy-silylpropyloxy)-2-hydroxy}-phenyl]-6-(4-methoxyphenyl)-1,3,5-triazine, 2,4-bis-[{4-(2′-methylpropenyloxy)-2-hydroxy}-phenyl]-6-(4-methoxyphenyl)-1,3,5-triazine, 2,4-bis-[{4-(1′,1′,1′,3′5′,5′,5′-heptamethylsiloxy-2′-methyl-propyloxy)-2-hydroxy}-phenyl]-6-(4-methoxyphenyl)-1,3,5-triazine, 2-(4-diethylamino-2-hydroxybenzoyl)-benzoic acid-hexylester (Uvinul® A Plus), indanylidene compounds as described in DE 100 55 940 (═WO 02/38537).

UV absorbers particularly suitable for combination are selected from the group comprising: p-aminobenzoic acid, 3-(4′-trimethylammonium)-benzylidene-bornan-2-one methylsulfate homomethyl salicylate (Neo Heliopan®HMS), 2-hydroxy-4-methoxy-benzophenone (Neo Heliopan®BB), 2-phenylbenzimidazolesulfonic acid (Neo Heliopan®Hydro), terephthalylidenedibornanesulfonic acid and salts (Mexoryl®SX), 4-tert-butyl-4′-methoxydibenzoylmethane (Neo Heliopan®357), 3-(4′-sulfo)benzylidene-bornan-2-one and salts, 2-ethylhexyl 2-cyano-3,3-diphenylacrylate (Neo Heliopan®303), N-[(2 and 4)-[2-(oxoborn-3-ylidene)methyl]benzyl]-acrylamide polymer, 2-ethylhexyl p-methoxycinnamate (Neo Heliopan®AV), ethyl p-aminobenzoate (25 mol) ethoxylated, isoamyl p-methoxycinnamate (Neo Heliopan®E1000), 2,4,6-trianilino-(p-carbo-2′-ethylhexyl-1′-ox y)-1,3,5-triazine (Uvinul®T150), phenol, 2-(2H-benzotriazol-2-yl)-4-methyl-6-(2-methyl-3(1,3,3,3-tetr amethyl-1-(trimethylsilyl)-oxy)-disiloxyanyl)-propyl), (Mexoryl®XL), bis-(2-ethylhexyl) 4,4′-[(6-[4-(1,1-dimethyl)-aminocarbonyl)-phenylamino]-1,3,5-triazine-2,4-diyl)-diimino]-bis-(benzoate), (UvasorbHEB), 3-(4′-methylbenzylidene)-d,l-camphor (Neo Helipan®MBC), 3-benzylidenecamphor, 2-ethylhexyl salicylate (Neo Heliopan®OS), 2-ethylhexyl 4-dimethylaminobenzoate (Padimate O), hydroxy-4-methoxybenzophenone-5-sulfonic acid and Na salt, 2,2′-methylene-bis-(6-(2H-benztriazol-2-yl)-4-1, 1,3,3-tetramethylbutyl)-phenol), (Tinosorb®M), phenylene-bis-benzimidazyltetrasulfonic acid disodium salt (Neo Heliopan®AP), 2,4-bis-[{(4-(2-ethyl-hexyloxy)-2-hydroxy}-phenyl]-6-(4-methoxyphenyl)-1,3,5-triazine, (Tinosorb®S), benzylidene malonate-polysiloxane (Parsol®SLX), menthyl anthranilate (Neo Heliopan®MA), hexyl 2-(4-diethylamino-2-hydroxybenzoyl)benzoate (Uvinul® A Plus), indanylidene compounds as described in DE 100 55 940 (=WO 02/38537).

Advantageous inorganic light protection pigments are selected from the group comprising finely dispersed metal oxides and metal salts, preferably titanium dioxide, zinc oxide (ZnO), iron oxide (e.g. Fe₂O₃), aluminum oxide (Al₂O₃); ceroxide (e.g. Ce₂O₃), manganese oxide (e.g. MnO), zirconium oxide (ZrO₂), silicon oxide (SiO₂), mixed oxides of the corresponding metals and mixtures of such oxides, barium sulfate and zinc stearate. Particular preference is for TiO₂- or zinc oxide-based pigments. In preferred embodiments the particles have an average diameter of less than 100 nm, preferably of between 5 and 50 nm and in particular preferably between 15 and 30 nm. They may have a spherical form, but particles may also be used which have an elliptical form or one that otherwise deviates from the spherical design. The pigments can also be surface-treated, e.g. rendered hydrophilic or hydrophobic. Typical examples are coated titanium dioxides, such as titanium dioxide T 805 (Degussa) or Eusolex® T2000 (Merck) or coated zinc oxide, such as e.g. zinc oxide NDM. As hydrophobic coatings here, above all silicone and in particular trialkoxyoctysilane or simethicone come into consideration. In sunscreens so-called micro- or nanopigments are preferably used.

The total quantity of inorganic pigments, in particular hydrophobic inorganic micropigments, in the inventive cosmetic or pharmaceutical, preferably dermatological, preparations is preferably in the range from 0.1 to 30% by weight, preferably 0.1 to 10.0, in particular 0.5 to 6.0% by weight, with reference to the total weight of the inventive cosmetic and pharmaceutical, preferably dermatological, preparations.

Inventive cosmetic and pharmaceutical, preferably dermatological, preparations can also contain one, two, three, four or more active substances and active substance combinations to combat skin ageing and wrinkles. In accordance with the invention all suitable or normal active substances to combat skin ageing and wrinkles used in cosmetic and pharmaceutical, preferably dermatological applications can be used. Advantageous active substances to combat ageing of the skin and wrinkles are selected from the group comprising Soya protein or protein hydrolysates, Soya isoflavones, hydrolysed rice protein, hydrolysed hazelnut protein, oligopeptides from hydrolysed Hibiscus esculentus extract, wheat protein, β-glucans, e.g. from oats, and derivatives thereof, glycoproteins, ursolic acid and its salts, betulin, betulinic acid and its salts, retinol, retinol palmitate, propyl gallate, precocenene, 6-hydroxy-7-methoxy-2,2-dimethyl-1 (2H)-benzopyran, 3,4-dihydro-6-hydroxy-7-methoxy-2,2-dimethyl-1 (2H)-benzopyran, creatine, or other synthetic or natural active substances for combating skin ageing and wrinkling, it also being possible for the latter to be used in the form of an extract of plants such as green tea, Rubus fruticosus, Sanguisorba officinalis, Centella asiatica, Ribes nigrum, Passiflora incarnata, Filipendula ulmaria, Phyllanthus emblica, Potentilla species, okra, algae, evening primrose, pomegranate, lady's mantel, rosemary, sage, Echinacea, birch, apple or Soya.

β-glucans are particularly preferably used as one, two, three, four or more other active substances for combating skin ageing; 1,3-1,4-linked β-glucan from oats, Rubus fruticosus extract or wheat protein is very particularly preferred.

Inventive cosmetic and pharmaceutical, preferably dermatological, preparations can also contain one, two, three, four or more other active substances, which stimulate skin and hair tinting or tanning by chemical or natural means. Particularly preferred here are substrates or substrate analogs of tyrosinase such as L-tyrosine, L-DOPA or L-dihydroxyphenylalanine, stimulators of tyrosinase activity or expression such as theophylline, caffeine, proopiomelanocortin peptides such as ACTH, alpha-MSH, peptide analogs thereof and other substances which bind to the melanocortin receptor, peptides such as Val-Gly-Val-Ala-Pro-Gly, Lys-IIe-Gly-Arg-Lys or Leu-IIe-Gly-Lys, purines, pyrimidines, folic acid, copper salts such as copper gluconate, chloride or pyrrolidonate, flavanoids, flavanon glycosides such as naringin and hesperidin, melanin derivatives such as Melasyn-100 and MelanZe, diacyl glycerols, aliphatic or cyclic diols, psoralens, prostaglandins and analogs thereof, activators of adenylate cyclase and compounds which activate the transfer of melanosomes into keratinocytes such as serine proteases or agonists of the PAR-2 receptor, extracts of plants and plant parts of the chrysanthemum species, sanguisorba species, walnut extracts, urucum extracts, rhubarb extracts, erythrulose and dihydroxyacetone.

Inventive cosmetic and pharmaceutical, preferably dermatological, preparations can also be used in combination with one, two, three, four or more skin lightening active substances. All skin-lightening active ingredients that are suitable for or commonly used for cosmetic and pharmaceutical, preferably dermatological, applications can be used here according to the invention. Advantageously one, two, three, four or more skin-lightening active substances are selected from the group comprising kojic acid (5-hydroxy-2-hydroxymethyl-4-pyranone), kojic acid derivatives such as e.g. kojic acid dipalmitate, arbutin, ascorbic acid, ascorbic acid derivatives, hydroquinone, hydroquinone derivatives, resorcinol, sulfur-containing molecules such as e.g. cysteine, alpha-hydroxy acids (e.g. citric acid, lactic acid, malic acid) and derivatives thereof, N-acetyl tyrosine and derivatives, undecenoyl phenylalanine, gluconic acid, 4-alkyl resorcinols, 4-(1-phenylethyl)1,3-benzene diol, chromone derivatives such as aloesin, flavonoids, thymol derivatives, 1-aminoethyl phosphinic acid, thio urea derivatives, ellagic acid, nicotinamide, zinc salts such as e.g. zinc chloride or gluconate, thujaplicin and derivatives, triterpenes such as maslinic acid, sterols such as ergosterol, benzofuranones such as senkyunolide, vinyl and ethyl guiacol, inhibitors of nitrogen oxide synthesis, such as e.g. L-nitroarginine and derivatives thereof, 2,7-dinitroindazole or thiocitrulline, metal chelators (e.g. alpha.-hydroxy fatty acids, palmitic acid, phytic acid, lactoferrin, humic acid, bile acid, bile extracts, bilirubin, biliverdin, EDTA, EGTA and derivatives thereof), retinoids, Soya milk, serine protease inhibitors or lipoic acid or other synthetic or natural active ingredients for skin and hair lightening, wherein the latter can also be used in the form of an extract from plants, such as e.g. bearberry extract, rice extract, licorice root extract or constituents concentrated therefrom, such as glabridin or licochalcone A, Artocarpus extract, extract from Rumex and Ramulus species, extracts from pine species (Pinus) and extracts from Vitis species or stilbene derivatives concentrated therefrom, extract of Saxifraga, mulberry, Scutelleria or/and grapes.

Inventive cosmetic and pharmaceutical, preferably dermatological, preparations can advantageously also contain one, two, three, four or more regulators. As moisture regulators (“moisturizers”) preferably one, two, three, four or more of the following substances are used: sodium lactate, urea, urea derivatives, alcohols, glycerol, diols such as propylene glycol, 1,2-pentanediol, 1,2-hexanediol and 1,2-octanediol and mixtures containing at least two diols, collagen, elastin or hyaluric acid, diacyl adipates, petroleum jelly, urocanic acid, lecithin, panthenol, phytanetriol, lycopene, (pseudo)ceramides, glycosphingolipids, cholesterol, phytosterols, chitosan, chondroitin sulfate, lanolin, lanolin esters, amino acids, alpha-hydroxy acids (e.g. citric acid, lactic acid, malic acid) and derivatives thereof, mono-, di- and oligosaccharides such as e.g. glucose, galactose, fructose, mannose, fruit sugars and lactose, poly sugars such as beta-glucanes, in particular 1,3-1,4-beta-glucane from oats, alpha-hydroxy fatty acids, triterpene acids such as betulinic acid or ursolic acid and algal extracts.

Inventive cosmetic and pharmaceutical, preferably dermatological, preparations can also be used with one, two, three, four or more osmolytes. As osmolytes preferably one, two, three, four or more of the following compounds are selected: substances from the group of sugar alcohols (myo-inositol, mannitol, sorbitol), quaternary amines such as taurine, choline, betaine, betaine glycine, ectoine, diglycerol phosphate, phosphorylcholine, glycerophosphorylcholines, amino acids such as glutamine, glycine, alanine, glutamate, aspartate or proline, phosphatidylcholine, phosphatidylinositol, inorganic phosphates, and polymers of the cited compounds such as proteins, peptides, polyamino acids and polyols. All osmolytes also have a skin-moistening action.

Inventive cosmetic and pharmaceutical, preferably dermatological, preparations can advantageously also contain one, two, three, four or more vitamins and vitamin precursors, in which vitamins and vitamins that are suitable and normal for cosmetic and pharmaceutical, preferably dermatological, applications can be used. As vitamins and vitamin precursors the following can preferably be mentioned: vitamin A (retinol) and its derivatives (e.g. vitamin A acetate, vitamin A acid, vitamin A aldehyde, vitamin A palmitate, vitamin A propionate), vitamin B1 (thiamine) and its salts (e.g. vitamin B1 hydrochloride, vitamin B1 mononitrate, thiamine diphosphate, thiamine pyrophosphate), vitamin B12 (cobalamin), vitamin B2 (vitamin G, riboflavin) and its derivatives (e.g. vitamin B2 tetraacetate), vitamin B3 and its derivatives (e.g. nicotinamide ascorbate, nicotinamide glycollate, nicotinamide hydroxycitrate, nicotinamide lactate, nicotinamide malate, nicotinamide mandelate, nicotinamide salicylate, nicotinamide thioctate), vitamin B4 (adenine) and its derivatives (e.g. adenine riboside, disodium flavin adenine dinucleotide, nicotinamide adenine dinucleotide), provitamin B5, vitamin B5 (pantothenic acid) and its derivatives (e.g. acetyl pantothenyl ethyl ether, allantoin calcium pantothenate, allantoin DL-pantothenyl alcohol, bis(pantothenamidoethyl) disulfide, calcium pantothenate, hydroxyethyl pantothenamide MEA, sodium pantothenate, N-D-pantothenoyl-2-(2-aminoethoxy)ethanol, N-D-pantothenoyl-2-aminoethanol, N-hydroxyethoxyethyl pantothenamide, N-hydroxyethyl pantothenamide, pantothenamide MEA, pantothenol, pantothenic acid lactone, pantothenic acid polypeptide, pantothenyl ethyl ether), vitamin B6 (pyridoxol, pyroxidal, pyridoxamine) and its derivatives (e.g. pyridoxine dicaprylate, vitamin B6 dilaurate, vitamin B6 dioctanoate, vitamin B6 dipalmitate, pyridoxine glycyrrhetinate, vitamin B6 hydrochloride, vitamin B6 phosphate, vitamin B6 serine, vitamin B6 tripalmitate), vitamin C (ascorbic acid) and its derivatives (e.g. 3-O-ethyl ascorbic acid, allantoin ascorbate, aminopropyl ascorbyl phosphate, araboascorbic acid, monosodium salt, ascorbic acid palmitate, ascorbic acid polypeptide, ascorbosilane C, ascorbyl dipalmitate, ascorbyl glucoside, ascorbyl inositol nicotinate, ascorbyl linoleate, ascorbyl methylsilanol pectinate, ascorbyl nicotinamide, ascorbyl phosphate magnesium, ascorbyl stearate, ascorbyl tetraisopalmitate, ascorbyl tocopheryl maleate, calcium ascorbate, chitosan ascorbate, D-arabino-ascorbic acid, disodium ascorbyl sulfate, glucosamine ascorbate, inositol hexanicotinate hexa-ascorbate, isoascorbic acid, L-ascorbic acid, 2-(dihydrogen phosphate), trisodium salt, L-ascorbic acid, 2-[(3-cholest-5-en-3-yl hydrogen phosphate], monosodium salt, L-ascorbic acid, 2-O-D-glucopyranosyl-, L-ascorbic acid, 3-O-ethyl ether, magnesium ascorbate, magnesium ascorbylborate, methoxy PEG-7 ascorbic acid, methylsilanol ascorbate, potassium ascorbyl tocopheryl phosphate, potassium ascorbylborate, sodium ascorbate, sodium ascorbyl phosphate, sodium ascorbyl/cholesteryl phosphate, sodium isoascorbate, sodium L-ascorbyl 2-phosphate, tetrahexyldecyl ascorbate), provitamin D, vitamin D (calciol) and its derivatives (e.g. vitamin D2, vitamin D3), vitamin E (D-alpha-tocopherol) and its derivatives (e.g. di-alpha-tocopherol, polyoxypropylene/polyoxyethylene/tocopherol ether, tocopherol cysteamine, tocopherol phosphate, sodium vitamin E phosphate, vitamin E acetate, vitamin E linoleate, vitamin E nicotinate, vitamin E succinate), vitamin F (essential fatty acids, linolenic acid and linoleic acid) and its derivatives (e.g. vitamin F ethyl ester, vitamin F glyceryl ester), vitamin H (vitamin B7, biotin), vitamin K1 (phylloquinone, phytonadione) and vitamin K3 (menadione, menaquinone).

Inventive cosmetic and pharmaceutical, preferably dermatological, preparations can likewise contain one, two, three, four or more additional plant extracts, which are normally produced by extraction from the entire plant, but in some cases also just from the flowers and/or leaves, wood, bark or roots of the plants. With regard to the plant extracts used, particular reference is made to the extracts that are listed in the table beginning on page 44 of the 3^{rd} edition of the “Leiffaden zur Inhaltsstoffdeklaration kosmetischer Mittel” (Guidelines on the declaration of the contents of cosmetic agents”), published by the Industrieverband Korperpflegemittel und Waschmittel e.V. (IKW), Frankfurt. Particularly advantageous are extracts of aloe, algae, apple, apricot, Arnica, avocado, birch, stinging nettle, mulberry, Calendula, ivy, Hibiscus, oak bark, strawberry, spruce, honeysuckle, barley, Ginkgo, ginseng, pomegranate, grapefruit, cucumber, oats, witch hazel, restharrow, henna, raspberry, elder, honeybush, hops, coltsfoot, kiwi, burdock, coconut, lavender, lime, linden, mallow, almond, mango, box holly, Melissa, olive, orange, peppermint, Pueraria, wild thyme, rooibos, rose, rosemary, horse chestnut, sage, sandalwood, yarrow, horsetail, Sophora, licorice, dead nettle, tea (green, white, black), thyme, grape, juniper, willow, rose-bay willow-herb, hawthorn, wheat, lady's smock, cinnamon, lemon and lemongrass. Particular preference here is for extracts of Aloe vera, algae, Arnica, stinging nettle, Calendula, witch hazel, linden, ginseng, cucumber, rosemary and sage. Mixtures of two or more plant extracts can also be used. Extraction agents which can be used for the preparation of the plant extracts mentioned are, inter alia, water, alcohols and mixtures thereof. In this context, among the alcohols lower alcohols, such as ethanol and isopropanol, and also polyhydric alcohols, such as ethylene glycol, propylene glycol and butylene glycol, are preferred, and in particular both as the sole extraction agent and in mixtures with water. The plant extracts can be employed both in the pure and in the diluted form.

Inventive cosmetic and pharmaceutical, preferably dermatological, preparations can advantageously also contain one, two, three, four or more cooling agents. Preference is for the following cooling: l-menthol, d-menthol, racemic menthol, menthone glycerine acetal, menthyl lactate, substituted menthyl-3-carboxylic acid amides (e.g. menthyl-3-carboxylic acid-N-ethylamide), 2-isopropyl-N-2,3-trimethyl butanamide, substituted cyclohexane carboxylic acid amides, 3-menthoxypropane-1,2-diol, 2-hydroxyethyl menthyl carbonate, 2-hydroxypropyl menthyl carbonate, N-acetyl glycine menthyl ester, isopulegol, menthyl hydroxycarboxylic acid esters (e.g. menthyl-3-hydroxybutyrate), monomenthyl succinate, 2-mercaptocyclodecanone, menthyl-2-pyrrolidin-5-one carboxylate, 2,3-dihydroxy-p-menthane, 3,3,5-trimethyl cyclohexanone glycerine ketal, 3-menthyl-3,6-di- and trioxaalkanoates, 3-menthyl methoxyacetate, icilin.

The inventive cosmetic and pharmaceutical, preferably dermatological, preparations can moreover also contain one, two, three, four or more perspiration-inhibiting active compounds (antiperspirants) and odor absorbers. Perspiration-inhibiting active compounds which are employed are, preferably, aluminum salts, such as aluminum chloride, aluminum hydrochloride, nitrate, sulfate, acetate etc. In addition, however, the use of compounds of zinc, magnesium and zirconium may also be advantageous. For use in cosmetic and pharmaceutical, preferably dermatological, antiperspirants, the aluminum salts and—to a somewhat lesser extent—aluminum/zirconium salt combinations have essentially proved suitable. The aluminum hydroxychlorides which are partly neutralized and therefore tolerated better by the skin, but not quite so active, are additionally worth mentioning. Alongside aluminum salts, further substances are also possible, such as, for example, a) protein-precipitating substances, such as, inter alia, formaldehyde, glutaraldehyde, natural and synthetic tannins and trichloroacetic acid, which bring about blockage of the sweat glands on the surface, b) local anesthetics (inter alia dilute solutions of e.g. lidocaine, prilocalne or mixtures of such substances), which eliminate sympathetic supply of the sweat glands by blockade of the peripheral nerve pathways, c) zeolites of the X, A or Y type, which, alongside the reduction in secretion of perspiration, also function as adsorbents for bad odors, and d) botulinus toxin (toxin of the bacterium Chlostridium botulinum), which is also employed in cases of hyperhidrosis, a pathologically increased secretion of perspiration, and the action of which is based on an irreversible blocking of the release of the transmitter substance acetylcholine, which is relevant for secretion of perspiration.

Odor absorbers are, for example, the laminar silicates described in DE 40 09 347, and of these in particular montmorillonite, kaolinite, nontronite, saponite, hectorite, bentonite and smectite, and furthermore, for example, zinc salts of ricinoleic acid. These likewise include deodorants, bactericidal or bacteriostatic deodorizing substances, such as e.g. hexachlorophene, 2,4,4′-trichloro-2′ hydroxydiphenyl ether (Irgasan), 1,6-di-(4-chlorophenylbiguanido)-hexane (chlorhexidine) and 3,4,4′-trichlorocarbanilide, as well as the active agents described in DE 37 40 186, DE 39 38 140, DE 42 04 321, DE 42 29 707, DE 42 29 737, DE 42 37 081, DE 43 09 372 and DE 43 24 219, and cationic substances, such as e.g. quaternary ammonium salts, and odor absorbers, such as e.g. Grillocin® (combination of zinc ricinoleate and various additives) or triethyl citrate, optionally in combination with ion exchange resins.

In various cases it may also be advantageous to employ inventive cosmetic and pharmaceutical, preferably dermatological, formulations in combination with one, two, three, four or more substances which are chiefly employed for inhibition of the growth of undesirable microorganisms on or in animal organisms. In this respect, alongside conventional preservatives, further active compounds which are worth mentioning, alongside the large group of conventional antibiotics, are, in particular, the products relevant for cosmetics, such as triclosan, climbazole, zinc pyrithione, ichthyol, Octopirox (1-hydroxy-4-methyl-6-(2,4,4-trimethylpentyl)-2(1H)-pyridone, 2-aminoethanol), chitosan, farnesol, octoxyglycerol, glycerol monolaurate, arylalkyl alcohols, such as e.g. phenylethyl alcohol, 3-phenyl-1-propanol, vetikol or muguet alcohol, polyglycerol esters, such as e.g. polyglyceryl 3-caprylates, and aliphatic diols, such as e.g. 1,2-decanediol, or combinations of the substances mentioned, which are employed, inter alia, against underarm odor, foot odor or dandruff formation.

Inventive cosmetic and pharmaceutical, preferably dermatological, preparations can in numerous cases also advantageously comprise one, two, three, for or more preservatives. Preservatives which are preferably chosen here are those such as benzoic acid and its esters and salts, propionic acid and its esters and salts, salicylic acid and its esters and salts, 2,4-hexadienoic acid (sorbic acid) and its esters and salts, formaldehyde and paraformaldehyde, 2-hydroxybiphenyl ether and its salts, 2-zinc-sulfidopyridine N-oxide, inorganic sulfites and bisulfites, sodium iodate, chlorobutanolum, 4-ethylmercury-(II)₅-amino-1,3-bis(2-hydroxybenzoic acid), its salts and esters, dehydracetic acid, formic acid, 1,6-bis(4-amidino-2-bromophenoxy)-n-hexane and its salts, the sodium salt of ethylmercury-(II)-thiosalicylic acid, phenylmercury and its salts, 10-undecylenic acid and its salts, 5-amino-1,3-bis(2-ethylhexyl)-5-methyl-hexahydropyrimidine, 5-bromo-5-nitro-1,3-dioxane, 2-bromo-2-nitro-1,3-propanediol, 2,4-dichlorobenzyl alcohol, N-(4-chlorophenyl)-N′-(3,4-dichlorophenyl)-urea, 4-chloro-m-cresol, 2,4,4′-trichloro-2′-hydroxy-diphenyl ether, 4-chloro-3,5-dimethylphenol, 1,1′-methylene-bis(3-(1-hydroxymethyl-2,4-dioximidazolidin-5-yl)urea), poly-(hexamethylenediguanide) hydrochloride, 2-phenoxyethanol, hexamethylenetetramine, 1-(3-chloroallyl)-3,5,7-triaza-1-azonia-adamantane chloride, 1-(4-chlorophenoxy)-1-(1H-imidazol-1-yl)-3,3-dimethyl-2-butanone, 1,3-bis-(hydroxy-methyl)-5,5-dimethyl-2,4-imidazolidinedione, benzyl alcohol, Octopirox, 1,2-dibromo-2,4-dicyanobutane, 2,2′-methylene-bis(6-bromo-4-chlorophenol), bromochlorophene, mixture of 5-chloro-2-methyl-3(2H)— isothiazolinone and 2-methyl-3(2H)-isothiazolinone with magnesium chloride and magnesium nitrate, 2-benzyl-4-chlorophenol, 2-chloroacetamide, chlorhexidine, chlorhexidine acetate, chlorhexidine gluconate, chlorhexidine hydrochloride, 1-phenoxy-propan-2-ol, N-alkyl(C₁₂-C₂₂)trimethyl-ammonium bromide and chloride, 4,4-dimethyl-1,3-oxazolidine, N-hydroxymethyl-N-(1,3-di(hydroxymethyl)-2,5-dioxoimidazolidin-4-yl)-N′-hydroxy-methylurea, 1,6-bis(4-amidino-phenoxy)-n-hexane and its salts, glutaraldehyde, 5-ethyl-1-aza-3,7-dioxabicyclo(3.3.0)octane, 3-(4-chlorophenoxy)-1,2-propanediol, hyamines, alkyl-(C₈-C₁₈)-dimethyl-benzyl-ammonium chloride, alkyl-(C₈-C₁₈)-dimethyl-benzylammonium bromide, alkyl-(C₈-C₁₈)-dimethyl-benzyl-ammonium saccharinate, benzyl hemiformal, 3-iodo-2-propynyl butylcarbamate, sodium hydroxymethyl-aminoacetate or sodium hydroxymethyl-aminoacetate.

Inventive cosmetic and pharmaceutical, preferably dermatological, formulations can also comprise one, two, three, four or more anionic, cationic, nonionic and/or amphoteric surfactants, especially if crystalline or microcrystalline solids, for example inorganic micropigments, are to be incorporated into the formulations. Surfactants are amphiphilic substances which can dissolve organic, nonpolar substances in water. In this context, the hydrophilic contents of a surfactant molecule are usually polar functional groups, for example —COO⁻, —OSO₃²⁻ or —SO₃⁻, while the hydrophobic parts as a rule are nonpolar hydrocarbon radicals. Surfactants are in general classified according to the nature and charge of the hydrophilic molecular moiety. A distinction can be made between four groups here:
anionic surfactants;
cationic surfactants;
amphoteric surfactants and
nonionic surfactants.

Anionic surfactants as a rule contain carboxylate, sulfate or sulfonate groups as functional groups. In aqueous solution, they form negatively charged organic ions in an acid or neutral medium. Cationic surfactants are almost exclusively characterized by the presence of a quaternary ammonium group. In aqueous solution, they form positively charged organic ions in an acid or neutral medium. Amphoteric surfactants contain both anionic and cationic groups and accordingly behave like anionic or cationic surfactants in aqueous solution, depending on the pH. In a strongly acid medium they have a positive charge, and in an alkaline medium a negative charge. On the other hand, they are zwitter-ionic in the neutral pH range. Polyether chains are typical of nonionic surfactants. Nonionic surfactants do not form ions in an aqueous medium.

### A. Anionic Surfactants

Anionic surfactants which are advantageously to be used are acylamino acids (and salts thereof), such as
acyl glutamates, for example sodium acyl glutamate, di-TEA-palmitoyl aspartate and sodium caprylic/capric glutamate;
acyl peptides, for example palmitoyl hydrolysed milk protein, sodium cocoyl hydrolysed Soya protein and sodium/potassium cocoyl hydrolysed collagen;
sarcosinates, for example myristoyl sarcosine, TEA-lauroyl sarcosinate, sodium lauroyl sarcosinate and sodium cocoyl sarcosinate;
taurates, for example sodium lauroyl taurate and sodium methylcocoyl taurate;
acyl lactylates, lauroyl lactylate and caproyl lactylate;
alaninates;
   carboxylic acids and derivatives, such as for example, lauric acid, aluminum stearate, magnesium alkanolate and zinc undecylenate;
ester-carboxylic acids, for example calcium stearoyl lactylate, laureth-6 citrate and sodium PEG-4 lauramide carboxylate;
ether-carboxylic acids, for example sodium laureth-13 carboxylate and sodium PEG-6 cocamide carboxylate;
   phosphoric acid esters and salts, such as, for example, DEA-oleth-10 phosphate and dilaureth-4 phosphate;
   sulfonic acids and salts, such as
acyl isethionates, e.g. sodium/ammonium cocoyl isethionate;
alkylarylsulfonates;
alkylsulfonates, for example sodium coconut monoglyceride sulfate, sodium C-₁₂₋₁₄ olefin-sulfonate, sodium lauryl sulfoacetate and magnesium PEG-3 cocamide sulfate;
sulfosuccinates, for example dioctyl sodium sulfosuccinate, disodium laureth-sulfosuccinate, disodium laurylsulfosuccinate and disodium undecylenamido-MEA-sulfosuccinate;
   and
   sulfuric acid esters, such as
alkyl ether-sulfate, for example sodium, ammonium, magnesium, MIPA and TIPA laureth sulfate, sodium myreth sulfate and sodium C12-13 pareth sulfate;
alkyl sulfates, for example sodium, ammonium and TEA lauryl sulfate.

### B. Cationic Surfactants

Cationic surfactants which are advantageously to be used are
alkylamines;
alkylimidazoles;
ethoxylated amines and
quaternary surfactants
   RNH₂CH₂CH₂COO⁻ (at pH=7)
   RNHCH₂CH₂COO— B⁺ (at pH=12) B⁺=any desired cation, e.g. Na⁺
ester quats;

Quaternary surfactants contain at least one N atom which is covalently bonded to 4 alkyl or aryl groups. This leads to a positive charge, independently of the pH. Alkylbetaine, alkylamidopropylbetaine and alkylamidopropylhydroxysulfaine are advantageous. The cationic surfactants used can furthermore preferably be chosen from the group consisting of quaternary ammonium compounds, in particular benzyltrialkyl-ammonium chlorides or bromides, such as, for example, benzyldimethylstearyl-ammonium chloride, furthermore alkyltrialkylammonium salts, for example cetyltrimethylammonium chloride or bromide, alkyldimethylhydroxy-ethylammonium chlorides or bromides, dialkyldimethylammonium chlorides or bromides, alkylamidoethyltrimethyl-ammonium ether-sulfates, alkylpyridinium salts, for example lauryl- or cetylpyrimidinium chloride, imidazoline derivatives and compounds having a cationic character, such as amine oxides, for example alkyldimethylamine oxides or alkylaminoethyldimethylamine oxides. Cetyltrimethyl-ammonium salts in particular are advantageously to be used.

### C. Amphoteric Surfactants

Amphoteric surfactants which are advantageously to be used are
acyl-/dialkylethylenediamine, for example sodium acylamphoacetate, disodium acylamphodipropionate, disodium alkylamphodiacetate, sodium acylamphohydroxy-propylsulfonate, disodium acylamphodiacetate and sodium acylamphopropionate;
N-alkylamino acids, for example aminopropyl alkylglutamide, alkylaminopropionic acid, sodium alkylimidodipropionate and lauroamphocarboxyglycinate.

### D. Nonionic Surfactants

Nonionic surfactants which are advantageously to be used are
alcohols;
alkanolamides, such as cocamides MEA/DEA/MIPA;
amine oxides, such as cocoamidopropylamine oxide;
esters which are formed by esterification of carboxylic acids with ethylene oxide, glycerol, sorbitan or other alcohols;
ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and coconut glycoside;
sucrose esters and ethers;
polyglycerol esters, diglycerol esters, monoglycerol esters;
methylglucose esters, esters of hydroxy acids.

The use of a combination of one, two, three, four or more anionic and/or amphoteric surfactants with one or more nonionic surfactants is furthermore advantageous.

The surface-active substance can be present in a total quantity of between 1 and 98% by weight in the formulations according to the invention, based on the total weight of the formulations.

Inventive cosmetic and pharmaceutical, preferably dermatological, formulations can also be in the form of emulsions.

The oily phase can advantageously be chosen from the following substance group:
mineral oils, mineral waxes;
fatty oils, fats, waxes and other natural and synthetic fat substances, preferably esters of fatty acids with alcohols of low C number, e.g. with isopropanol, propylene glycol or glycerol, or esters of fatty alcohols with alkanoic acids of low C number or with fatty acids;
alkyl benzoates;
silicone oils, such as dimethylpolysiloxanes, diethylpolysiloxanes, diphenylpolysiloxanes and mixed forms thereof.

Compounds which can advantageously be employed are (a) esters of saturated and/or unsaturated branched and/or unbranched alkanecarboxylic acids having a chain length of from 3 to 30 C atoms and saturated and/or unsaturated, branched and/or unbranched alcohols having a chain length of from 3 to 30 C atoms, (b) esters of aromatic carboxylic acids and saturated and/or unsaturated, branched and/or unbranched alcohols having a chain length of from 3 to 30 C atoms. Preferred ester oils are isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 3,5,5-trimethylhexyl 3,5,5-trimethylhexanoate, 2-ethylhexyl isononanoate, 2-ethylhexyl 3,5,5-trimethylhexanoate, 2-ethylhexyl 2-ethylhexanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of such esters, e.g. jojoba oil.

The oily phase can furthermore advantageously be chosen from the group consisting of branched and unbranched hydrocarbons and waxes, silicone oils and dialkyl ethers, the group consisting of saturated or unsaturated, branched or unbranched alcohols, and the fatty acid triglycerides, namely the triglycerol esters of saturated and/or unsaturated, branched and/or unbranched alkanecarboxylic acids having a chain length of from 8 to 24, in particular 12 to 18 C atoms. The fatty acid triglycerides can advantageously be chosen from the group consisting of synthetic, semi-synthetic and natural oils, e.g. olive oil, sunflower oil, Soy oil, groundnut oil, rapeseed oil, almond oil, palm oil, coconut oil, palm kernel oil and more of the like. Any desired blends of such oil and wax components can also advantageously be employed. In some cases it is also advantageous to employ waxes, for example cetyl palmitate, as the sole lipid component of the oily phase, and the oily phase is advantageously chosen from the group which consists of 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride and dicaprylyl ether. Mixtures of C₁₂₋₁₅-alkyl benzoate and 2-ethylhexyl isostearate, mixtures of C₁₂₋₁₅-alkyl benzoate and isotridecyl isononanoate and mixtures of C₁₂₋₁₅-alkyl benzoate, 2-ethylhexyl isostearate and isotridecyl isononanoate are particularly advantageous. The hydrocarbons paraffin oil, squalane and squalene can also advantageously be used. The oily phase can furthermore have a content of cyclic or linear silicone oils or consist entirely of such oils, it nevertheless being preferable to use an additional content of other oily phase components in addition to the silicone oil or silicone oils. Cyclomethicone (e.g. decamethylcyclopentasiloxane) can advantageously be employed as a silicone oil. However, other silicone oils, for example undecamethylcyclotrisiloxane, polydimethylsiloxane and poly(methyl-phenylsiloxane), can also advantageously be used. Mixtures of cyclomethicone and isotridecyl isononanoate and of cyclomethicone and 2-ethylhexyl isostearate are furthermore particularly advantageous.

The aqueous phase of inventive cosmetic and dermatological preparations which are in the form of an emulsion, which can contain the inventive 3-(4-hydroxy-3-methoxyphenyl)-1-(4-hydroxyphenyl)-1-propanone of formula 1, can advantageously comprise: alcohols, diols or polyols of low C number and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and furthermore alcohols of low C number, e.g. ethanol, isopropanol, 1,2-propanediol and glycerol, and, in particular, one or more thickening agents, which can advantageously be chosen from the group consisting of silicon dioxide, aluminum silicates, polysaccharides and derivatives thereof, e.g. hyaluronic acid, xanthan gum and hydroxypropyl-methylcellulose, particularly advantageously from the group consisting of polyacrylates, preferably a polyacrylate from the group consisting of the so-called Carbopols, for example Carbopols of the types 980, 981, 1382, 2984 and 5984, in each case individually or in combination.

Inventive cosmetic and pharmaceutical, preferably dermatological, preparations in the form of an emulsion advantageously comprise one, two, three, four or more emulsifiers. O/W emulsifiers can advantageously be chosen, for example, from the group consisting of polyethoxylated or polypropoxylated or polyethoxylated and polypropoxylated products, e.g.:
the fatty alcohol ethoxylates;
the ethoxylated wool wax alcohols;
the polyethylene glycol ethers of the general formula R—O—(—CH₂—CH₂—O—)ₙ—R′;
the fatty acid ethoxylates of the general formula R—COO—(—CH₂—CH₂—O—)ₙ—H;
the etherified fatty acid ethoxylates of the general formula R—COO—(—CH₂—CH₂—O—)ₙ—R′;
the esterified fatty acid ethoxylates of the general formula R—COO—(—CH₂—CH₂—O—)ₙ—C(O)—R′;
the polyethylene glycol glycerol fatty acid esters;
the ethoxylated sorbitan esters;
the cholesterol ethoxylates;
the ethoxylated triglycerides;
the alkyl ether-carboxylic acids of the general formula R—COO—(—CH₂—CH₂—O—)ₙ—OOH, wherein n represents a number from 5 to 30;
the polyoxyethylene sorbitol fatty acid esters;
the alkyl ether-sulfates of the general formula R—O—(—CH₂—CH₂—O—)ₙ—SO₃—H;
the fatty alcohol propoxylates of the general formula R—O—(—CH₂—CH(CH₃)—O—)ₙ—H;
the polypropylene glycol ethers of the general formula R—O—(—CH₂—CH(CH₃)—O—)ₙ—R′;
the propoxylated wool wax alcohols;
the etherified fatty acid propoxylates R—COO—(—CH₂—CH(CH₃)—O—)ₙ—R′;
the esterified fatty acid propoxylates of the general formula R—COO—(—CH₂—CH(CH₃)—O—)ₙ—C(O)—R′;
the fatty acid propoxylates of the general formula R—COO—(—CH₂—CH(CH₃)—O—)ₙ—H;
the polypropylene glycol glycerol fatty acid esters
the propoxylated sorbitan esters;
the cholesterol propoxylates;
the propoxylated triglycerides;
the alkyl ether-carboxylic acids of the general formula R—O—(—CH₂—CH(CH₃)—O—)ₙ—CH₂—COOH;
the alkyl ether-sulfates and the acids on which these sulfates are based of the general formula R—O—(—CH₂—CH(CH₃)—O—)ₙ—SO₃—H;
the fatty alcohol ethoxylates/propoxylates of the general formula R—O—Xₙ—Yₘ—H;
the polypropylene glycol ethers of the general formula R—O—Xₙ—Yₘ—R′;
the etherified fatty acid propoxylates of the general formula R—COO—Xₙ—Yₘ—R′;
the fatty acid ethoxylates/propoxylates of the general formula R—COO—Xₙ—Yₘ—H.

According to the invention, the one, two, three, four or more polyethoxylated or polypropoxylated or polyethoxylated and polypropoxylated 0/W emulsifiers employed are particularly advantageously chosen from the group consisting of substances having HLB values of 11-18, very particularly advantageously having HLB values of 14.5-15.5, if the 0/W emulsifiers contain saturated radicals R and R′. If the 0/W emulsifiers contain unsaturated radicals R and/or R′, or isoalkyl derivatives are present, the preferred HLB value of such emulsifiers can also be lower or higher.

It is of advantage to choose the fatty alcohol ethoxylates from the group consisting of ethoxylated stearyl alcohols, cetyl alcohols and cetyl stearyl alcohols (cetearyl alcohols). The following are particularly preferred:

polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetyl stearyl ether (ceteareth-13), polyethylene glycol (14) cetyl stearyl ether (ceteareth-14), polyethylene glycol (15) cetyl stearyl ether (ceteareth-15), polyethylene glycol (16) cetyl stearyl ether (ceteareth-16), polyethylene glycol (17) cetyl stearyl ether (ceteareth-17), polyethylene glycol (18) cetyl stearyl ether (ceteareth-18), polyethylene glycol (19) cetyl stearyl ether (ceteareth-19) and polyethylene glycol (20) cetyl stearyl ether (ceteareth-20).

It is furthermore advantageous to choose the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

Sodium laureth-11 carboxylate can advantageously be used as an ethoxylated alkyl ether-carboxylic acid or salt thereof. Sodium laureth 1-4 sulfate can advantageously be used as an alkyl ether-sulfate. Polyethylene glycol (30) cholesteryl ether can advantageously be used as an ethoxylated cholesterol derivative. Polyethylene glycol (25) sojasterol has also proved suitable.

The polyethylene glycol (60) evening primrose glycerides can advantageously be used as ethoxylated triglycerides.

It is furthermore advantageous to choose the polyethylene glycol glycerol fatty acid esters from the group consisting of polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caproate, polyethylene glycol (20) glyceryl oleate, polyethylene glycol (20) glyceryl isostearate and polyethylene glycol (18) glyceryl oleate/cocoate.

It is likewise favorable to choose the sorbitan esters from the group consisting of polyethylene glycol (20) sorbitan monolaurate, polyethylene glycol (20) sorbitan monostearate, polyethylene glycol (20) sorbitan monoisostearate, polyethylene glycol (20) sorbitan monopalmitate and polyethylene glycol (20) sorbitan monooleate.

Advantageous W/O emulsifiers which can be employed are: fatty alcohols having 8 to 30 carbon atoms, monoglycerol esters of saturated and/or unsaturated, branched and/or unbranched alkanecarboxylic acids having a chain length of from 8 to 24, in particular 12 to 18 C atoms, diglycerol esters of saturated and/or unsaturated, branched and/or unbranched alkanecarboxylic acids having a chain length of from 8 to 24, in particular 12 to 18 C atoms, monoglycerol ethers of saturated and/or unsaturated, branched and/or unbranched alcohols having a chain length of from 8 to 24, in particular 12 to 18 C atoms, diglycerol ethers of saturated and/or unsaturated, branched and/or unbranched alcohols having a chain length of from 8 to 24, in particular 12 to 18 C atoms, propylene glycol esters of saturated and/or unsaturated, branched and/or unbranched alkanecarboxylic acids having a chain length of from 8 to 24, in particular 12 to 18 C atoms and sorbitan esters of saturated and/or unsaturated, branched and/or unbranched alkanecarboxylic acids having a chain length of from 8 to 24, in particular 12 to 18 C atoms.

W/O emulsifiers which are advantageous in particular are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaproate and glyceryl monocaprylate.

Inventive cosmetic and pharmaceutical, preferably dermatological, preparations for topical prophylactic or cosmetic treatment of the skin can regularly comprise a high content of care substances. According to a preferred embodiment, the compositions comprise one or more animal and/or plant fats and oils having care properties, such as olive oil, sunflower oil, refined Soy oil, palm oil, sesame oil, rapeseed oil, almond oil, borage oil, evening primrose oil, coconut oil, shea butter, jojoba oil, oat oil, sperm oil, beef tallow, neat's foot oil and lard, and optionally further care constituents, such as, for example, fatty alcohols having 8-30 C atoms. The fatty alcohols here can be saturated or unsaturated and linear or branched. Alcohols which can be employed are, for example, decanol, decenol, octanol, octenol, dodecanol, dodecenol, octadienol, decadienol, dodecadienol, oleyl alcohol, ricinoleyl alcohol, erucyl alcohol, stearyl alcohol, isostearyl alcohol, cetyl alcohol, lauryl alcohol, myristyl alcohol, arachidyl alcohol, caprylyl alcohol, capryl alcohol, linoleyl alcohol, linolenyl alcohol and behenyl alcohol, and Guerbet alcohols thereof, it being possible for the list to be extended virtually as desired by further alcohols of related structural chemistry. The fatty alcohols preferably originate from natural fatty acids, being conventionally prepared from the corresponding esters of the fatty acids by reduction. Fatty alcohol fractions which are formed by reduction from naturally occurring fats and fatty oils, such as e.g. beef tallow, groundnut oil, colza oil, cottonseed oil, Soy oil, sunflower oil, palm kernel oil, linseed oil, maize oil, castor oil, rape oil, sesame oil, cacao butter and coconut fat, can furthermore be employed.

Care substances which can be combined in an outstanding manner with formulations according to the invention moreover also include
waxes, such as e.g. candelilla wax or carnauba wax;
ceramides, where ceramides are understood as meaning N-acylsphingosins (fatty acid amides of sphingosin) or synthetic analogs of such lipids (so-called pseudo-ceramides), which significantly improve the water retention capacity of the stratum corneum;
phospholipids, for example Soy lecithin, egg lecithin and cephalins;
Vaseline, paraffin oils and silicone oils; the latter include, inter alia, dialkyl- and alkylarylsiloxanes, such as dimethylpolysiloxane and methylphenylpolysiloxane, as well as alkoxylated and quaternized derivatives thereof.

The inventive method for cosmetic treatment or prevention of inflammatory processes of the skin is preferably characterized in that the inventive cosmetic or pharmaceutical preparation is applied to one or more parts of the skin which exhibit an inflammation and/or no inflammation.

The compound of formula 1 to be used in accordance with the invention can preferably be obtained by a manufacturing process comprising the following steps:

(a) conversion (aldol condensation) of p-hydroxyacetophenone with vanillin to 3-(4-hydroxy-3-methoxyphenyl)-1-(4-hydroxyphenyl)-2-propen-1-one;
(b) reduction, preferably hydrogenation, of the reaction product obtained in step (a).

The unsaturated ketone 3-(4-hydroxy-3-methoxyphenyl)-1-(4-hydroxyphenyl)-2-propen-1-one is known from the literature, e.g. from DE 1 099 732, DE 1 447 016 or also DE 2 256 961.

Preferred embodiments and other aspects of the present invention are shown by the attached claims and the following examples, wherein the examples do not restrict the invention. Unless otherwise stated, all data refer to weight.

### EXAMPLE 1

### Synthesis of 3-(4-hydroxy-3-methoxyphenyl)-1-(4-hydroxyphenyl)-1-propanone, Compound of Formula 1

(a) Conversion of p-Hydroxyacetophenone with Vanillin:

20 g of potassium hydroxide are placed in 100 g of diethylene glycol diethyl ether, heated to 120° C. with agitation and then a mixture of 14 g of p-hydroxyacetophenone and 15 g vanillin is added within 1 hour. Once the dosing is complete agitation is continued for a further 20 minutes, followed by hydrolysis and adjustment of the pH to between 6 and 7. Following phase separation the solvent is removed and 25 g 3-(4-hydroxy-3-methoxyphenyl)-1-(4-hydroxyphenyl)-2-propen-1-one are obtained with a yield of 93% of the theoretical amount.

### (b) Hydrogenation of the Reaction Product Obtained in Step (a):

10 g of 3-(4-hydroxy-3-methoxyphenyl)-1-(4-hydroxyphenyl)-2-propen-1-one are dissolved in 100 g of tetrahydrofuran, 0.2 g Pd on activated charcoal are added (Pd content: 5% by weight, water content approx. 50% by weight, in each case with reference to the total mass of the catalyst) and hydrogenated under normal pressure at ambient temperature (approx. 20° C.). Following removal of the catalyst and the solvent 9 g of 3-(4-hydroxy-3-methoxyphenyl)-1-(4-hydroxyphenyl)-propan-1-one are obtained. Yield: 89% of theoretical amount.

Spectroscopic data of 3-(4-hydroxy-3-methoxyphenyl)-1-(4-hydroxyphenyl)-propan-1-one (formula 1):

¹³C-NMR (CDCl₃; 75.5 MHz): 6 (ppm)=197.42 (s), 161.85 (s), 147.24 (s), 144.44 (s), 132.03 (s), 130.37 (d), 130.37 (d), 128.18 (s), 120.27 (d), 115.11 (d), 115.07 (d), 115.07 (d), 112.53 (d), 55.42 (q), 39.30 (t), 29.42 (t);

MS: m/z (%)=M⁺-Ion 272(82), 151(24), 137(77), 121(100), 93(19), 65(22).

### EXAMPLE 2-11

### Cosmetic Formulations Containing 3-(4-hydroxy-3-methoxyphenyl)-1-(4-hydroxyphenyl)-1-propanone of Formula 1

Formulations containing inventive 3-(4-hydroxy-3-methoxyphenyl)-1-(4-hydroxyphenyl)-1-propanone with an anti-inflammatory effect.

In the following Table 2:

1=Skin-lightening day cream O/W
2=Skin-soothing lotion with plant extracts O/W
3=Aftersun balm
4=Body spray
5=Sunscreen lotion (O/W), broad-band protection
6=W/O night cream

### 7=Shampoo

8=Self-tanning cream
9=Barrier repair cream O/W
10=Antiperspirant/deodorant roll-on

TABLE 2
RAW MATERIAL NAME
(PRODUCER)
3-(4-hydroxy-3-

| methoxyphenyl)- | | FORMULATION | | | | | | | | | |
|---|---|---|---|---|---|---|---|---|---|---|---|
| 1-(4-hydroxyphenyl)- | | 1 | 2 | 3 | 4 | 5 | 6 | 7 | 8 | 9 | 10 |
| 1-propanone | | WEIGHT % | | | | | | | | | |
| (Formula 1) | INCI | 0.3 | 0.01 | 0.5 | 0.1 | 0.3 | 0.2 | 0.05 | 0.2 | 1.0 | 0.1 |
| Abil 350 | Dimethicone | 0.5 | 2.0 | 1.0 | | | | | 0.5 | 0.5 | |
| (Degussa- | | | | | | | | | | | |
| Goldschmidt) | | | | | | | | | | | |
| Allantoin (Merck) | Allantoin | | 0.2 | 0.1 | | | | | | | |
| Aloe Vera Gel | Water (Aqua), | | | 3.0 | | | 3.0 | | | | |
| Concentrate | Aloe Barbadensis | | | | | | | | | | |
| 10/1 (Symrise) | Leaf Juice | | | | | | | | | | |
| Alugel 34 TH | Aluminum Stearate | | | | | | 1.0 | | | | |
| (Baerlocher) | | | | | | | | | | | |
| Aqua-Ceramide | Cetyloxypropyl Glyceryl | | 0.1 | | | | | | | | 0.1 |
| (Kao) | Methoxypropyl | | | | | | | | | | |
| | Myristamide | | | | | | | | | | |
| Arbutin | β-Arbutin | 1.0 | | | | | | | | | |
| (Sabinsa) | | | | | | | | | | | |
| Sodium Ascorbyl | Sodium Ascorbyl | 2.0 | | 1.0 | | | | | | | |
| Phosphate | Phosphate | | | | | | | | | | |
| (EMD | | | | | | | | | | | |
| Chemicals) | | | | | | | | | | | |
| -(-Alpha-)- | Bisabolol | 0.3 | 0.4 | | | | | 0.05 | | 0.5 | |
| Bisabolol, | | | | | | | | | | | |
| natural | | | | | | | | | | | |
| (Symrise) | | | | | | | | | | | |
| Butylene Glycol | Butylene Glycol | | | 5.0 | | | | | | | |
| Carbopol ETD | Carbomer | | | | | 0.2 | | | | | |
| 2050 (Noveon) | | | | | | | | | | | |
| Carbopol Ultrez- | Carbomer | | 0.1 | | | | | | | | |
| 10 (Noveon) | | | | | | | | | | | |
| Ceramide 2 | Ceramide 2 | 0.1 | | | | | | | | | |
| (Sederma) | | | | | | | | | | | |
| Ceramide | Hydroxypropyl | | | | | 0.1 | | | | | |
| PC104 (Pacific | Bispalmitamide MEA | | | | | | | | | | |
| Corporation) | | | | | | | | | | | |
| Ceramide SL | Hydroxyethyl Palmityl | | | | | | 0.1 | | | | |
| (Sino Lion) | Oxyhydroxypropyl | | | | | | | | | | |
| | Palmitamide | | | | | | | | | | |
| Cetiol OE | Dicaprylyl Ether | | | 4.0 | | | | | | | |
| (Cognis) | | | | | | | | | | | |
| Cetiol SB 45 | Butyrospermum Parkii | | | 1.0 | | | | | | | |
| (Cognis) | (Shea Butter) | | | | | | | | | | |
| Citric Acid 10% | Citric Acid | | | | | | | 0.3 | | | |
| sol. | | | | | | | | | | | |
| Comperlan 100 | Cocamide MEA | | | | | | | 0.5 | | | |
| (Cognis) | | | | | | | | | | | |
| Dihydroxyacetone | Dihydroxyacetone | | | | | | | | 5.0 | | |
| (Merck) | | | | | | | | | | | |
| Dow Corning | Cyclohexasiloxane and | | | | | 2.0 | | | | | |
| 246 Fluid (Dow | Cyclopentasiloxane | | | | | | | | | | |
| Corning) | | | | | | | | | | | |
| Dow Corning | Cyclomethicone | | | | 0.5 | | | | | | |
| 345 Fluid (Dow | | | | | | | | | | | |
| Corning) | | | | | | | | | | | |
| D-Panthenol | Panthenol | | | 1.0 | | | | | | | |
| (BASF) | | | | | | | | | | | |
| Dracorin CE | Glyceryl Stearate Citrate | 5.0 | | | | | | | 5.0 | 1.5 | |
| (Symrise) | | | | | | | | | | | |
| Dracorin GMS | Glyceryl Stearate | | 2.0 | | | | | | | 2.0 | |
| (Symrise) | | | | | | | | | | | |
| Dracorin GOC | Glyceryl Oleate Citrate, | | | | 2.0 | | | | | | |
| (Symrise) | Caprylic/Capric | | | | | | | | | | |
| | Triglyceride | | | | | | | | | | |
| Drago-Beta- | Water (Aqua), Butylene | 0.3 | | | | | | | | | |
| Glucan | Glycol, Glycerin, Avena | | | | | | | | | | |
| (Symrise) | Sativa (Oat), Kernel | | | | | | | | | | |
| | Extract | | | | | | | | | | |
| Dragocid Liquid | Phenoxyethanol, | | 0.8 | 0.7 | | 0.7 | 0.8 | | | 0.8 | |
| (Symrise) | Methylparaben, | | | | | | | | | | |
| | Ethylparaben, | | | | | | | | | | |
| | Butylparaben, | | | | | | | | | | |
| | Propylparaben, | | | | | | | | | | |
| | Isobutylparaben | | | | | | | | | | |
| Dragoderm | Glycerin, Triticum | | | | | | | 2.0 | | | |
| (Symrise) | Vulgare (Wheat) Gluten, | | | | | | | | | | |
| | Water (Aqua) | | | | | | | | | | |
| Drago-Oat- | Water (Aqua), Butylene | | | | 1.0 | | | | | | |
| Active (Symrise) | Gylcol, Avena Sativa | | | | | | | | | | |
| | (Oat) Kernel Extract | | | | | | | | | | |
| Dragosan W/O | Polyglyceryl-3- | | | | | | 1.0 | | | | |
| Liquid (Symrise) | Polyricinoleate, Sorbitan | | | | | | | | | | |
| | Isostearate | | | | | | | | | | |
| Dragosan W/O P | Sorbitan Isostearate, | | | | | | 6.0 | | | | |
| (Symrise) | Hydrogenated Castor | | | | | | | | | | |
| | Oil, Ceresin, Beeswax | | | | | | | | | | |
| | (Cera Alba) | | | | | | | | | | |
| Dragoxat EH | Ethylhexy | 3.0 | 3.0 | | 4.0 | | | | 3.0 | | |
| (Symrise) | Ethylhexanoate | | | | | | | | | | |
| Dragoxat 89 | Ethylhexyl isononanoate | | | | | | | | | 2.0 | |
| (Symrise) | | | | | | | | | | | |
| EDETA B | Tetrasodium EDTA | | | | | | | 0.1 | | | |
| Powder (BASF) | | | | | | | | | | | |
| EDETA DB | Disodium EDTA | | | | | 0.1 | | | 0.1 | | |
| (BASF) | | | | | | | | | | | |
| Emulsiphos | Potassium Cetyl | | 2.0 | | | 1.5 | | | | 2.0 | |
| (Symrise) | Phosphate, | | | | | | | | | | |
| | Hydrogenated Palm | | | | | | | | | | |
| | Glycerides | | | | | | | | | | |
| Ethanol 96% | Ethanol | | | | | | | | 2.0 | | 30.0 |
| Extrapone | Glycerin, Water (Aqua), | | 0.2 | | | | | | | | |
| Green Tea GW | Camellia Sinensis Leaf | | | | | | | | | | |
| (Symrise) | Extract | | | | | | | | | | |
| Extrapone Witch | Propylene Glycol, | | | | | | 1.0 | | | | |
| Hazel Distillate | Hamamelis Virginiana | | | | | | | | | | |
| colorless | (Witch Hazel) Water, | | | | | | | | | | |
| (Symrise) | Water (Aqua), | | | | | | | | | | |
| | Hamamelis Virginiana | | | | | | | | | | |
| | (Witch Hazel) Extract | | | | | | | | | | |
| Extrapone | Glycerin, Water (Aqua), | | 0.3 | | | | | | | 0.5 | |
| Rosemary GW | Rosmarinus officinalis | | | | | | | | | | |
| (Symrise) | (Rosemary) Leaf Extract | | | | | | | | | | |
| Farnesol | Farnesol | | | | | | | | | | 0.5 |
| (Symrise) | | | | | | | | | | | |
| Frescolat ML | Menthyl Lactate | | | 0.8 | | | | | | | |
| crist. (Symrise) | | | | | | | | | | | |
| Genapol LRO | Sodium Laureth Sulfate | | | | | | | 37.0 | | | |
| liquid (Cognis) | | | | | | | | | | | |
| Givobio GZN | Zinc Gluconate | | | | | | | | | 0.5 | |
| (Seppic) | | | | | | | | | | | |
| Glycerin 85% | Glycerin | 3.0 | 2.0 | 4.0 | | 4.7 | 2.0 | | 1.5 | 3.0 | |
| Hydrolite-5 | Pentylene Glycol | | | | 5.0 | | | | 3.5 | | |
| (Symrise) | | | | | | | | | | | |
| Hydroviton | Water, Glycerin, Sodium | | | | | | | | | 1.0 | |
| (Symrise) | Lactate, TEA Lactate, | | | | | | | | | | |
| | Serine, Lactic Acid, | | | | | | | | | | |
| | Urea, Sorbitol, Sodium | | | | | | | | | | |
| | Chloride, | | | | | | | | | | |
| | Lauryl | | | | | | | | | | |
| | Diethylenediaminoglycine, | | | | | | | | | | |
| | Lauryl | | | | | | | | | | |
| | Aminopropylglycine, | | | | | | | | | | |
| | Allantoin | | | | | | | | | | |
| Ingwer CO₂ | Zingiber Officinale | 0.003 | | | | | | | | 0.01 | 0.001 |
| Extrakt (Flavex) | (Ginger) Root Extract | | | | | | | | | | |
| Irgasan DP 300 | Triclosan | | | | | | | | | | 0.3 |
| (Ciba Geigy) | | | | | | | | | | | |
| Isodragol | Triisononanoin | | 2.0 | | | | | | | 3.0 | |
| (Symrise) | | | | | | | | | | | |
| Isopropylpalmitat | Isopropyl Palmitate | 4.0 | | | | | | | 4.0 | | |
| (Symrise) | | | | | | | | | | | |
| Karion F (Merck) | Sorbitol | | | | | | 2.0 | | | | |
| Keltrol RD (CP- | Xanthan Gum | 0.2 | 0.1 | | | | | | | | |
| Kelco) | | | | | | | | | | | |
| Keltrol T (Danby- | Xanthan Gum | | | | | 0.2 | | | 0.3 | | |
| Chemie) | | | | | | | | | | | |
| Kojic acid | Kojic acid | 1.0 | | | | | | | | | |
| (Cosmetochem) | | | | | | | | | | | |
| Lanette 16 | Cetyl Alcohol | 1.0 | | | | | | | 1.0 | | |
| (Cognis) | | | | | | | | | | | |
| Lanette O | Cetearyl Alcohol | | 3.0 | | | 1.0 | | | | 2.0 | |
| (Cognis) | | | | | | | | | | | |
| Lara Care A-200 | Galactoarabinan | | | 0.3 | | | | | | | |
| (Rahn) | | | | | | | | | | | |
| Magnesium | Magnesium Chloride | | | | | | 0.7 | | | | |
| Chloride (Merck) | | | | | | | | | | | |
| Merquat 550 | Polyquaternium-7 | | | | | | | 0.5 | | | |
| (Ondeo Nalco) | | | | | | | | | | | |
| NAOH 10% sol. | Sodium Hydroxide | | | | | | | | | 0.3 | |
| Naringin | 4′,5,7-Trihydroxyflavon- | | | | | | | 0.5 | 2.0 | | |
| (Exquim) | 7-O-neohesperidoside | | | | | | | | | | |
| Sodium | Sodium Benzoate | | | | | | | 0.5 | | | |
| benzoate | | | | | | | | | | | |
| Natrosol 250 | Hydroxyethylcellulose | | | | | | | | | | 0.3 |
| HHR | | | | | | | | | | | |
| (Aqualon) | | | | | | | | | | | |
| Neo Heliopan | Butyl Methoxy- | | | | | 1.0 | | | | | |
| 357 (Symrise) | dibenzoylmethane | | | | | | | | | | |
| Neo Heliopan | Disodium Phenyl | | | | | 10 | | | | | |
| AP (Symrise) | Dibenzimidazole | | | | | | | | | | |
| (10% as sodium | Tetrasulfonate | | | | | | | | | | |
| salt) | | | | | | | | | | | |
| Neo Heliopan | Ethylhexyl | | | | | 3.0 | | | | | |
| AV (Symrise) | Methoxycinnamate | | | | | | | | | | |
| Neo Heliopan | Phenylbenzimidazole | | | | | 6.7 | | | | | |
| Hydro (Symrise) | Sulfonic Acid | | | | | | | | | | |
| (15% as sodium | | | | | | | | | | | |
| salt) | | | | | | | | | | | |
| Neo Heliopan | 4-Methylbenzylidene | | | | | 1.5 | | | | | |
| MBC (Symrise) | Camphor | | | | | | | | | | |
| Neo Heliopan | Ethylhexyl Salicylate | | | | | 5.0 | | | | | |
| OS (Symrise) | | | | | | | | | | | |
| Neutral Oil | Caprylic/Capric | 6.0 | | | 4.0 | 2.0 | | | 6.0 | 10.0 | |
| | Triglyceride | | | | | | | | | | |
| Oxynex 2004 | BHT | | | | | | 0.1 | | | | |
| (Merck) | | | | | | | | | | | |
| Paraffin oil 5 | Paraffinum Liquidum | | | | 4.0 | | | | | | |
| Grade E | | | | | | | | | | | |
| (Parafluid) | | | | | | | | | | | |
| PCL Liquid 100 | Cetearyl Ethylhexoate | 3.0 | 5.0 | | 7.0 | | | | | | |
| (Symrise) | | | | | | | | | | | |
| PCL Solid | Stearyl Heptanoate, | | 2.0 | | | | | | | | |
| (Symrise) | Stearyl Caprylate | | | | | | | | | | |
| PCL-Liquid | Cetearyl | | | | | | 12.0 | | 3.0 | | |
| (Symrise) | Ethylhexanoate, | | | | | | | | | | |
| | Isopropyl Myristate | | | | | | | | | | |
| Pemulen TR-2 | Acrylates/C10-30 Alkyl | | | 0.3 | 0.2 | | | | | | |
| (Noveon) | Acrylate Crosspolymers | | | | | | | | | | |
| 4-(1- | 4-(1-Phenylethyl)1,3- | 0.5 | | | | | | | | | |
| Phenylethyl)1,3- | benzenediol | | | | | | | | | | |
| benzenediol | | | | | | | | | | | |
| Propylene | Propylene Glycol | | 5.0 | | | | | | | | |
| Glycol-1,2 99P | | | | | | | | | | | |
| GC | | | | | | | | | | | |
| Pseudoceramide | N-(1-Hexadecanoyl)-4- | | 0.1 | | | | | 0.2 | | 0.5 | |
| 391 | hydroxy-L-prolin-(1- | | | | | | | | | | |
| | hexadecyl-ester | | | | | | | | | | |
| Retinyl Palmitate | Retinyl Palmitate | | | | | | 0.2 | | | | |
| in Oil | | | | | | | | | | | |
| (DSM Nutritional | | | | | | | | | | | |
| Products) | | | | | | | | | | | |
| Sepigel 305 | Polyacrylamides, C13- | | | | | | | | 1.0 | | |
| | 14 Isoparaffin, Laureth-7 | | | | | | | | | | |
| Sodium Chloride | Sodium Chloride | | | | | | | 1.0 | | | |
| Sodium | Sodium Hydroxide | | 0.3 | 0.6 | 0.4 | | | | | | |
| Hydroxide (10% | | | | | | | | | | | |
| sol. | | | | | | | | | | | |
| Solubilizer | PEG-40 Hydrogenated | | | | | | | | | | 2.0 |
| 611674 | Castor Oil, Trideceth-9, | | | | | | | | | | |
| (Symrise) | Water (Aqua) | | | | | | | | | | |
| Sun Flower Oil | Helianthus Annuus | | | | | | 5.0 | | | | |
| (Wagner) | (Sunflower) Seed Oil | | | | | | | | | | |
| Sweet Almond | Prunus dulcis | | | | | | 5.0 | | | | |
| Oil (Wagner) | | | | | | | | | | | |
| SymMatrix | Maltodextrin, Rubus | | 0.1 | | | 0.3 | 1.0 | | | | |
| (Symrise) | Fruticosus (Blackberry) | | | | | | | | | | |
| | Leaf Extract | | | | | | | | | | |
| Symdiol 68 | 1,2 Hexanediol, | 0.5 | | | | | | | | | |
| (Symrise) | Caprylylglycol | | | | | | | | | | |
| Symrise | Fragrance | 0.3 | 0.3 | 0.3 | 0.2 | 0.4 | 0.4 | 0.5 | 0.3 | 0.3 | 1.0 |
| Fragrance | | | | | | | | | | | |
| Tamasterol | Phytosterols | | | | | | | | | 0.3 | |
| (Tama | | | | | | | | | | | |
| Biochemicals) | | | | | | | | | | | |
| Tego Betain L7 | Cocamidopropyl Betain | | | | | | | 6.0 | | | |
| (Degussa) | | | | | | | | | | | |
| Tegosoft PC | | | | | | | | | | 0.3 | |
| 31 (Degussa) | | | | | | | | | | | |
| Tegosoft TN | C12-15 Alkyl Benzoate | | | 5.0 | | 5.0 | | | | | |
| (Degussa) | | | | | | | | | | | |
| Triethanolamine, | Triethanolamine | | | | | 0.5 | | | | | |
| 99% | | | | | | | | | | | |
| Tocopherol | Tocopheryl Acetate | | | 0.5 | | 0.5 | 3.0 | | | 0.3 | |
| Acetate (DSM | | | | | | | | | | | |
| Nutritional | | | | | | | | | | | |
| Products) | | | | | | | | | | | |
| Zirkonal L 450 | Aluminum Zirconium | | | | | | | | | | 37.0 |
| (BK Giulini) | Pentachlorohydrate | | | | | | | | | | |
| | (40% aqueous solution) | | | | | | | | | | |
| Water, | Water (Aqua) | ad | ad | ad | ad | ad | ad | ad | ad | ad | ad |
| demineralised | | 100 | 100 | 100 | 100 | 100 | 100 | 100 | 100 | 100 | 100 |

### SPECIFIC EMBODIMENTS

Specific embodiment one comprises a cosmetic or pharmaceutical preparation including or comprising the following components:
a) a quantity with an anti-inflammatory effect of a compound of formula 1

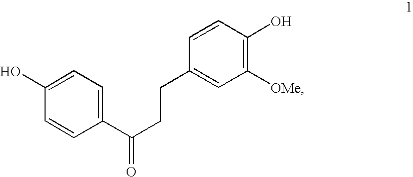

b) if necessary one, two, three, four or more additional compounds with an anti-inflammatory effect,
and
c) one or more cosmetic and/or pharmaceutical base or inactive ingredients or additives that are compatible with components (a) and (b).

Specific embodiment two comprises the preparation as in specific embodiment one, wherein in addition the preparation
d) includes an effective quantity of one, two, three, four or more UV filters, so that the sun protection factor of the cosmetic or pharmaceutical preparation is >2.

Specific embodiment three comprises the preparation as in specific embodiment one or two, wherein the preparation also
e) includes one, two, three, four or more compounds in a quantity in relation to the finished preparation, which in a preparation without the compound of formula 1 has an inflammatory effect on human or animal skin,
   in which the quantity of the compound of formula 1 with reference to the finished preparation, is sufficient to reduce, eliminate or suppress the inflammatory effect of the compounds from step e) on the skin.

Specific embodiment three comprises the preparation as in any one of specific embodiments one to three, wherein the preparation is selected from the group comprising liquid formulations, preferably cleaning solutions or impregnating liquids; semi-solid formulations, preferably ointments and crèmes.

Specific embodiment five comprises a compound of formula 1

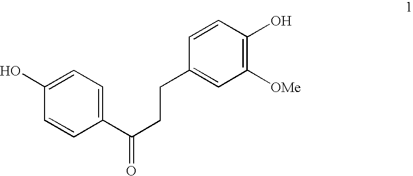

for use as a medicinal product.

Specific embodiment six comprises the compound of formula 1 as in specific embodiment five, wherein the medicinal product is applied topically.

Specific embodiment seven comprises the compound of formula 1 as in specific embodiment six, wherein the medicinal product is used for the treatment of inflammations of the skin.

Specific embodiment eight comprises a use of a compound of formula 1

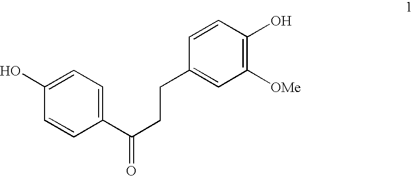

in a quantity with an anti-inflammatory effect for the treatment or prevention of inflammation.

Specific embodiment nine comprises a use of a compound of formula 1

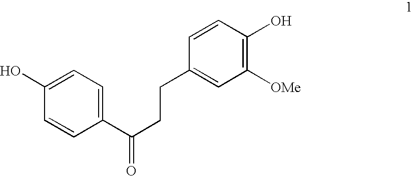

in a quantity with an anti-inflammatory effect in cosmetic preparations.

Specific embodiment ten comprises a use of a compound of formula 1

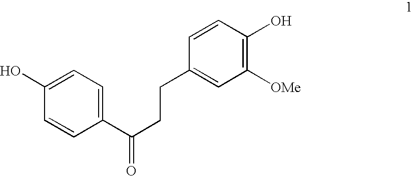

in a quantity with an anti-inflammatory effect for the production of a medicinal product for the treatment or prevention of inflammation.

Specific embodiment eleven comprises a use of a quantity with an anti-inflammatory effect of a compound of formula 1

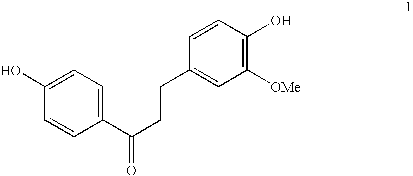

or a cosmetic or pharmaceutical preparation as in any one of specific embodiments one to four for the reduction, elimination or suppression of an inflammatory effect of one, two, three, four or more compounds on human or animal skin.

Specific embodiment twelve comprises a method for the cosmetic treatment or prevention of inflammatory processes of the skin including or comprising the topical application of a quantity with an anti-inflammatory effect of the preparation as claimed in any one of specific embodiments one to four to the skin of a human or an animal's body.

Specific embodiment thirteen comprises the method as in specific embodiment twelve, wherein the preparation is applied to one or more parts of the skin which exhibit inflammation and/or no inflammation.

Specific embodiment fourteen comprises a method for treating or preventing an inflammatory effect of one, two, three, four or more compounds on human or animal skin including or comprising the following steps:
a) provision of one, two, three, four or more compounds that have an inflammatory effect on the skin,
b) provision of a compound of formula 1

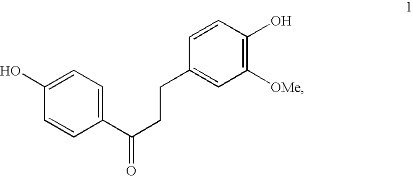

c) if necessary provision of one, two, three, four or more base or inactive ingredients and/or additives for cosmetic or pharmaceutical preparations and
d) mixing of the compounds from step a) with the compound of formula 1 from step b) and if necessary the base or inactive ingredients and/or additives from step c) to form a preparation,
   in which one or more compound(s) from step a) is/are included in a quantity in relation to the finished preparation, which in a preparation without the compound of formula 1 have an inflammatory effect on human or animal skin and
   in which the compound of formula 1 is included in a quantity that is sufficient to reduce, eliminate or suppress the inflammatory effect of the compounds from step a) on the skin.

Specific embodiment fifteen comprises a scent composition, including or comprising
a) a quantity of an aromatic substance or mixture of aromatic substances with a sensorial effect,
b) a quantity with an anti-inflammatory effect of a compound of formula 1

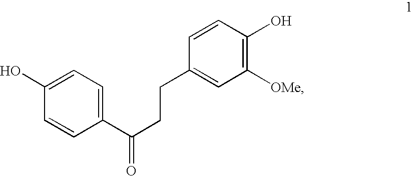

c) if necessary one, two, three, four or more additional compounds with an anti-inflammatory effect and
d) if necessary one or more base or inactive ingredients and/or additives.

It is claimed:

1. A cosmetic or pharmaceutical preparation including or comprising the following components:
a) a quantity with an anti-inflammatory effect of a compound of formula 1 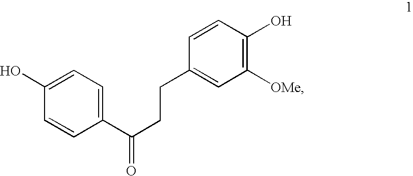
b) if necessary one, two, three, four or more additional compounds with an anti-inflammatory effect,
and
c) one or more cosmetic and/or pharmaceutical base or inactive ingredients or additives that are compatible with components (a) and (b).

2. The preparation as claimed in claim 1, wherein in addition the preparation
d) includes an effective quantity of one, two, three, four or more UV filters, so that the sun protection factor of the cosmetic or pharmaceutical preparation is >2.

3. The preparation as claimed in claim 1, wherein the preparation also
e) includes one, two, three, four or more compounds in a quantity in relation to the finished preparation, which in a preparation without the compound of formula 1 has an inflammatory effect on human or animal skin,
in which the quantity of the compound of formula 1 with reference to the finished preparation, is sufficient to reduce, eliminate or suppress the inflammatory effect of the compounds from step e) on the skin.

4. The preparation as claimed in claim 1, wherein the preparation is selected from the group comprising liquid formulations and semi-solid formulations.

5. A compound of formula 1 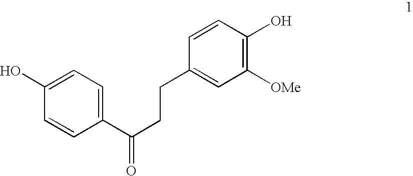
and a pharmaceutically acceptable carrier, formulated as a medicinal product.

6. The compound of formula 1 as claimed in claim 5, wherein the medicinal product is formulated for topical application.

7. The compound of formula 1 as claimed in claim 6, wherein the medicinal product is formulated for the treatment of inflammations of the skin.

8. A method for the treatment or prevention of inflammation comprising applying a compound of formula 1 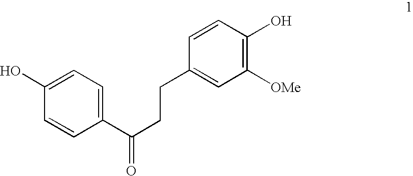
in a quantity with an anti-inflammatory effect.

9. A method for the preparation of a composition, comprising mixing a compound of formula 1 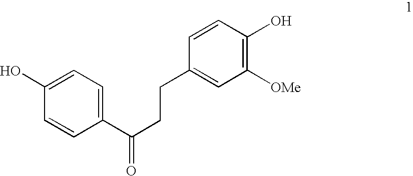
in a quantity having an anti-inflammatory effect in a cosmetic preparation.

10. A method for the production of a medicinal product for the treatment or prevention of inflammation, comprising combining a quantity of a compound of formula 1 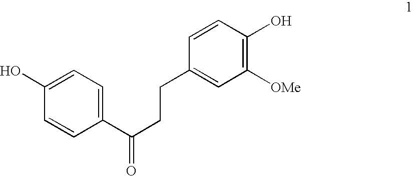
having an anti-inflammatory effect with a pharmaceutically acceptable carrier.

11. A method for the reduction, elimination or suppression of an inflammatory effect of one, two, three, four or more compounds on human or animal skin, comprising applying a quantity with an anti-inflammatory effect of a compound of formula 1 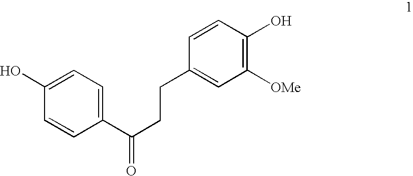
or a cosmetic or pharmaceutical preparation as claimed in claim 1.

12. A method for the cosmetic treatment or prevention of inflammatory processes of the skin including or comprising the topical application of a quantity with an anti-inflammatory effect of the preparation as claimed in claim 1 to the skin of a human or an animal's body.

13. The method as claimed in claim 12, wherein the preparation is applied to one or more parts of the skin which exhibit inflammation and/or no inflammation.

14. A method for treating or preventing an inflammatory effect of one, two, three, four or more compounds on human or animal skin including or comprising the following steps:
a) provision of one, two, three, four or more compounds that have an inflammatory effect on the skin,
b) provision of a compound of formula 1 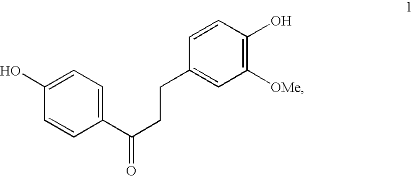
c) if necessary provision of one, two, three, four or more base or inactive ingredients and/or additives for cosmetic or pharmaceutical preparations and
d) mixing of the compounds from step a) with the compound of formula 1 from step b) and if necessary the base or inactive ingredients and/or additives from step c) to form a preparation,
in which one or more compound(s) from step a) is/are included in a quantity in relation to the finished preparation, which in a preparation without the compound of formula 1 have an inflammatory effect on human or animal skin and
in which the compound of formula 1 is included in a quantity that is sufficient to reduce, eliminate or suppress the inflammatory effect of the compounds from step a) on the skin.

15. A scent composition, including or comprising
a) a quantity of an aromatic substance or mixture of aromatic substances with a sensorial effect,
b) a quantity with an anti-inflammatory effect of a compound of formula 1 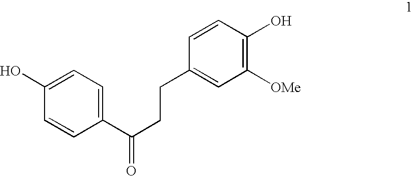
c) if necessary one, two, three, four or more additional compounds with an anti-inflammatory effect and
d) if necessary one or more base or inactive ingredients and/or additives.

16. The preparation as claimed in claim 4, wherein the liquid formulations are cleaning solutions or impregnating liquids.

17. The preparation as claimed in claim 4, wherein the semi-solid formulations are selected from the group comprising ointments and crèmes.
